(19) United States
(12) Patent Application Publication
(10) Pub. No.: US 2002/0049151 A1
(43) Pub. Date: Apr. 25, 2002

(54) Therapeutic approaches to diseases by suppression of the NURR subfamily of nuclear transcription factors

(76) Inventors: Evelyn Murphy, Dublin (IE); Orla M. Conneely, Houston, TX (US); Oliver Fitzgerald, Dublin (IE); Barry Bresnihan, Dublin (IE)

Correspondence Address: FULBRIGHT & JAWORSKI, LLP, 1301 MCKINNEY, SUITE 5100, HOUSTON, TX, 77010-3095, US

(21) Appl. No.: 09/853,386

(22) Filed: May 11, 2001

Related U.S. Application Data
(60) Provisional application No. 60/203,645, filed on May 12, 2000.

(51) Int. Cl. A61K 48/00; A61K 31/00

(52) U.S. Cl. 514/1; 514/44

(57) ABSTRACT

Synovial CRH functions in a paracrine manner to induce the nuclear transcription factor NURR1, which is abundantly expressed in the inflammatory cells of both rheumatoid arthritis and psoriatic arthritis synovium. This induction is suppressed by glucocorticoids. The invention is directed to the pivotal role the NURR subfamily of transcription factors plays in modulation of peripheral CRH and CRH-mediated signaling through the CRH-receptor subtype R1α, particularly in the inflammatory process in human arthritis.

This application claims priority to U.S. Provisional Patent Application Ser. No. 60/203,645, filed May 12, 2000.

The work herein was supported by grants from the United States Government. The Government may have certain rights in the invention.

### FIELD OF THE INVENTION

The present invention generally relates to the central role of the NURR subfamily of transcription factors in mediating multiple inflammatory signals. More particularly the invention relates to the nuclear receptors NURR1, NUR77 and NOR1 and their role in modulation of peripheral CRH and CRH-mediated signaling, which is an important component of inflammatory processes such as in human arthritis.

### BACKGROUND OF THE INVENTION

Many aspects of vertebrate development, differentiation and homeostasis are regulated by small molecular hormones and signaling molecules which control gene expression in a ligand-dependent manner through binding to nuclear receptors. These molecules include sex steroids, corticosteroids, thyroid hormones, and vitamin D3, many of which have been cloned. In spite of the diversity in ligands and biological functions, these receptors belong to the structurally and genetically related nuclear receptor superfamily. The common structural feature of this superfamily is a tripartite domain structure consisting of a hypervariable N-terminus which contributes to the transactivation function; a highly conserved DNA binding domain which is responsible for DNA recognition and dimerization; and the conserved C-terminus, which contains subdomains II and III, and is involved in nuclear localization, ligand binding, receptor dimerization, silencing and transactivation (see, e.g., Evans, 1988; O'Malley, 1990; Beato, 1991; and Tsai and O'Malley, 1994). The most conserved feature of this superfamily is the DNA binding domain (DBD) which contains 65-68 amino acid residues. Eight of the nine non-variant cysteines form two type II zinc modules. The sequence identity in the DBD of any member to the rest of family ranges between 40 to 99%. The high degree of conservation of this segment led to the discovery of many more structurally-related receptors in recent years, which are termed orphan receptors as the identity of ligands and physiological functions are unknown (see, e.g., O'Malle, 1988; Beato, 1991; Laudet et al., 1992; O'Malley and Conneely, 1992).

Orphan receptors form an important, though artificially grouped, subfamily of the nuclear superfamily. The majority of them were identified on the basis of homology in the DBD region to previously cloned members. Several strategies were adopted to search for new members including using the entire DBD as a probe to screen cDNA libraries at reduced stringency (Giguere et al., 1988; and Law et al., 1992), or RT-PCR amplification using degenerate primers coding the most conserved regions in the DBD (Schmidt et al., 1992; Andre et al., 1993), or using degenerate oligonucleotides corresponding to DBD consensus sequences to screen libraries (Issemann and Green, 1990; Chang and Kokontis, 1988).

Several lines of evidence indicate that members of the NURR subfamily play an important coordinate regulatory role at all levels of the HPA axis (Wilson et al., 1993; Murphy and Conneely 1997; Philips et al., 1997). The NURR subfamily belongs to a superfamily of structurally related transcription factors that control a variety of developmental and physiological processes. The family includes receptors for steroid hormones, vitamins and thyroid hormone as well as orphan receptors whose cognate ligand(s), if any, remain to be identified (Evans, 1998; O'Malley and Conneely, 1992). NURR1 (Nur-related factor 1; also called RNR-1 and NOT) is an orphan member of this superfamily that is expressed predominantly in the central nervous system (Law et al., 1992; Scearce et al., 1993; Mages et al., 1994). The protein exhibits a close structural relationship to the orphan receptors NUR77 (also called NGFI-β/N10/NAK) (Hazel et al., 1988; Milbrant, 1998; Ryseck et al., 1989; Nakai et al., 1990) and NOR-1 (also called MINOR/-TEC) (Ohkura et al., 1994; Maruyama et al., 1995; Hedvat and Irving, 1995). These three proteins comprise the NURR subfamily that bind to the same cis-acting consensus sequence (NBRE) to regulate target gene expression (Ohkura et al., 1994, Wilson et al., 1991; Murphy et al., 1995). Unlike most nuclear receptors the NURR subfamily are products of immediate early genes whose expression can be differentially induced in response to a variety of extracellular stimuli, including growth factors (Hazel et al., 1998; Milbrandt, 1998), neurotransmitters (Watson and Milbrandt, 1989) and polypeptide hormones (Wilson et al., 1993; Murphy and Conneely, 1997; Davis and Lau, 1994). NURR1 and NUR77 can regulate the expression of the CRH and POMC genes by interacting with specific cis-acting sequences in their proximal promoter region. NURR1 and NUR77 are rapidly induced by CRH in primary pituitary cells, resulting in increased synthesis of POMC (Murphy and Conneely, 1997). Glucocorticoid repression of the POMC gene is mediated by glucocorticoid receptor dependent inhibition of activation of the POMC gene by NURR1 and NUR77 (Evans, 1998; Philips et al., 1997). NOR-1 possesses an identical DNA binding domain and is capable of binding the same cis-acting consensus sequence which structurally groups the orphan receptor into the NURR subfamily. Therefore, the close structural relationship, the identical cis-acting consensus sequence, and the ability of the different members of the NURR subfamily of transcription factors to functionally complement one another are strong indications that the NURR subfamily members have redundancy of function.

Corticotropin Releasing Hormone (CRH), a major regulator of the hypothalamic pituitary axis (HPA), exerts significant anti-inflammatory effects predominantly through the immunosuppressive actions of glucocorticoids (Vale et al. 1989; Cato and Wade, 1996). Products of an activated immune system, including IL-1β, IL-6 and TNFα, act directly and indirectly to stimulate the synthesis and secretion of hypothalamic CRH (Tumball and Rivier, 1999). CRH exerts its functions through receptor-mediated activation of cyclic AMP (cAMP) pathways, which potently stimulate the synthesis of pro-opiomelanocorticotropin (POMC) (Aguilera et al., 1982). POMC is a precursor molecule of several neuropeptides including adrenocorticotropic hormone (ACTH), which is released from the pituitary and regulates the synthesis of adrenal glucocorticoids. To maintain homeostasis glucocorticoids inhibit CRH and POMC synthesis and secretion at the level of the hypothalamus and pituitary.

A growing body of data now supports direct involvement of extra-hypothalamic or immune CRH in the modulation of immune responses. A role for immune CRH in the mediation of the localized inflammatory response is supported in an in vivo rat model of acute inflammation where CRH is produced and is active locally (Karalis et al., 1991). In this rat model peripheral CRH, in contrast to its indirect immunosuppressive effect, is associated with a local pro-inflammatory autocrine/paracrine role. Immunoneutralization of this localized CRH synthesis, using anti-CRH antibodies, causes a specific suppression of the inflammatory response (Karalis et al., 1991). Most importantly, the construction of mice lacking CRH confirms that peripheral CRH is required for induction of the inflammatory response in vivo (Karalis et al., 1999). However, despite evidence that CRH is an important mediator of inflammatory reactions during systemic immune system activation, the regulation and mode of action of peripheral CRH remains to be established. The discovery that increased immunoreactive CRH is found in RA synovial tissue (Crofford et al., 1993; Nishioka et al., 1996) and in several animal models of inflammatory joint disease (Crofford et al., 1992; Webster et al., 1998) highlights the potential involvement of peripheral CRH in the pathogenesis of inflammatory arthritis. Given the importance of the NURR transcription factors in mediating hypothalamic CRH function (Murphy et al., 1993), the regulation of NURR1, NUR77 and NOR1 gene expression in synovium explants in response to CRH and pro- and anti-inflammatory mediators is demonstrated herein.

The study of orphan receptors has expanded our view of the superfamily. It has facilitated the discovery of new ligands, for Example, 9-cis-retionic acid has been identified as a ligand for the RXRs (retinoid-x receptors) (Mangelsdorf et al., 1990; Levin et al., 1992; Heyman et al., 1992). Alternate ligand-independent activation pathways have been discovered which include membrane receptor-activated phosphorylation pathways for the orphan receptors NUR77 and COUP-TF (Hazel et al., 1991; Power et al., 1991). Receptor isoforms, which add diversity to individual hormone function, have been found to be very common in this superfamily (Mangelsdorf et al., 1990; Levin et al., 1992; Heyman et al., 1992; Hazel et al., 1991; Power et al., 1991; Chen et al., 1993). Orphan receptors can also contribute to metabolic function (e.g. PPAR (peroxisome proliferator-activated receptor) subfamily) by regulating a key enzyme of the peroxisomal fatty acid β-oxidation system, the acyl-CoA oxidase gene in response to unsaturated fatty acids (Dreyer et al., 1992). Therefore, the cloning and characterization of orphan receptors has played a significant role in discovery of new signaling pathways and transactivation mechanisms.

It is well established that IL-1β and TNFα mediate transcription coupling through NF-κB, while PGE₂ signals by activation of CREB dependent pathways. Additionally, it is well established that CRH signals by activation of CREB dependent pathways. However, the role that CRH receptors play in the mediation of CREB dependent and NF-κB pathways remains undefined. Two distinct subtypes of CRH receptors, CRH-R1 and CRH-R2, have been isolated and characterized (Aguilera et al., 1987; Perrin et al., 1995) and are both pharamacologically distinct and unique in their expression patterns within the brain and in peripheral tissues. In healthy mice, CRH-R1 is limited primarily to regions of the brain including the brain stem, cerebellum, cerebral cortex, and medial septum and the pituitary gland. Because of this localization, mice deficient in CRH-R1 have been constructed and employed to study the specific role that CRH-R1 plays in postnatal development (U.S. Pat. 6,147,275, issued Nov. 14, 2000). The presence of a start codon in the a 5′—untranslated region of CRH-R1 has been implicated in the inhibition of mRNA translation and suggests that the upstream start codon plays a role in regulating translation of the CRH-R1 receptor (Xu et al., 2001). CRH-R2 is expressed in several peripheral tissues including the heart, skeletal muscle, gastrointestinal tract and the epididymis, and expression in the brain is concentrated in the lateral septum and hypothalamic areas. Partial agonists of CRH-R1 have been described for the treatment of stress related disorders (U.S. Pat. No. 6,127,399, issued Oct. 3, 2000).

Information elucidating the detailed biochemical processes involved in inflammatory immune disease is increasing, and the pathological result of such diseases is well established. Rheumatism is a chronic systemic inflammatory autoimmune disease that causes swelling and pain in the multi-joints and malaise, infirmity, weight loss, febricula and anorexia in other body organs. Criteria for the classification of rheumatism include morning stiffness, arthritis of 3 or more joint areas, arthritis of hand joints, symmetric arthritis, radiographic changes, serum rheumatoid factor and rheumatoid nodules. A patient is considered in the art to have rheumatism if he/she has satisfied at lease 4 of these 7 criteria. Corticosteroids are very important anti-inflammatory agents, suppressing the formation of several mediators of inflammation and articular cartilage degradative enzymes and, as such, effectively reducing the inflammation and pain associated with traumatic joint disease. Rheumatoid arthritis (RA) is a type of arthritis and a common cause of disability. After 12 years of disease, more than 80% of patients with RA are partially disabled, and 16% are completely disabled, and life expectancy is shortened by an average of 7 years in men and 3 years in women (Matteson, 2000). The most common causes of decreased life expectancy associated with RA are vasculitis, inflammation of the blood vessels; side effects of drugs, such as bleeding from a stomach ulcer; and an increased risk of infection, which is the a result of suppression of the immune system by the required drugs. The goals of therapy are to relieve pain, control inflammation, and prevent joint destruction, and includes disease-modifying antirheumatic drugs (DMARDs), such as gold, methotrexate, or tumor necrosis factor (TNF) antagonists. Recently, the Food and Drug Administration and the European Medicine Evaluation Agency approved of etanercept, a soluble TNFα type II receptor-IgG1 fusion protein, and infliximab, a chimeric monoclonal antibody against TNFα. The development of these drugs resulted from the increased understanding of the role that pro-inflammatory mediators play in rheumatoid arthritis. However, the detailed mechanisms of these complex cellular interactions remain unknown. Furthermore, the delivery of antagonists of the pro-inflammatory mediators by gene therapy poses a potential therapeutic tool for rheumatoid arthritis (Choy et al., 2001).

Inflammation and hyperplasia of the synovium are hallmarks of rheumatoid arthritis. The normal synovium is a delicate tissue lining the joint capsule; however, in RA, the synovium transforms into an aggressive, tumor-like structure called the pannus. Synoviocytes (fibroblasts) and macrophages within the synovium orchestrate a self-perpetuating inflammatory response via the autocrine/paracrine actions of cytokines (i.e. IL1β, TNFα and IL6). Proliferating synoviocytes in the vicinity of the affected cartilage produce matrix-degrading molecules, including matrix metalloproteinases (MMPs) and express growth factors and adhesion molecules. It is the persistent invasive and destructive growth of synovial tissue that ultimately leads to joint erosion. Many of the inflammatory cytokines and MMPs implicated in RA are regulated by inducible transcription factors. Transcription factors such as NFκB, AP1 and CREB are pivotal regulators of inflammatory responses. Several independent studies have implicated abnormal expression of these transcription factors in the modulation of gene expression known to regulate cellular proliferation, cytokine and MMP production in RA synovium.

The formation of active inflamed pannus is thought to be central to erosive disease resulting in joint destruction. Angiogenesis, the formation of new blood vessels, is one of the earliest histopathologic findings in rheumatoid arthritis and appears to be required for pannus development. New blood vessels are, as a major source of cytokine and protease activity, thought to be critical factors in the initiation and persistence of arthritic disease.

### SUMMARY OF THE INVENTION

In an embodiment of the present invention there is a method of treating an organism for an inflammatory immune disease comprising the step of reducing expression of a NURR subfamily nucleic acid sequence selected from the group consisting of SEQ ID NO:1, SEQ ID NO:47 and SEQ ID NO:76. In a specific embodiment, the reduction of the expression of the NURR subfamily nucleic acid sequence comprises inhibiting synthesis of a nucleic acid sequence of SEQ ID NO: 1. In another specific embodiment the inflammatory immune disease is selected from the group consisting of a chronic inflammatory joint disease, ulcerative colitis and thyroiditis. In a further specific embodiment the inflammatory joint disease is arthritis. In a further specific embodiment the arthritis is selected from the group consisting of rheumatoid arthritis, psoriatic arthritis and sarcoid arthritis.

In an embodiment of the present invention there is a method of treating an organism for an inflammatory immune disease comprising the step of reducing the level of a polypeptide comprising a NURR subfamily amino acid sequence selected from the group consisting of SEQ ID NO:33, SEQ ID NO: 64 and SEQ ID NO:91. In a specific embodiment, the reduction of the polypeptide comprises inhibiting amino acid synthesis of a sequence comprising SEQ ID NO:33. In another specific embodiment the inflammatory immune disease is selected from the group consisting of a chronic inflammatory joint disease, ulcerative colitis and thyroiditis. In a further specific embodiment the inflammatory joint disease is arthritis. In a further specific embodiment the arthritis is selected from the group consisting of rheumatoid arthritis, psoriatic arthritis and sarcoid arthritis.

In another embodiment of the present invention, there is a method of treating an organism for an inflammatory immune disease comprising the step of inhibiting transcriptional activity of a sequence selected from the group consisting of SEQ ID NO:33, SEQ ID NO:64 and SEQ ID NO:91. In a specific embodiment, the sequence is SEQ ID NO:33. In another specific embodiment the inflammatory immune disease is selected from the group consisting of a chronic inflammatory joint disease, ulcerative colitis and thyroiditis. In a further specific embodiment the inflammatory joint disease is arthritis. In a further specific embodiment the arthritis is selected from the group consisting of rheumatoid arthritis, psoriatic arthritis and sarcoid arthritis.

In an additional embodiment of the present invention there is an antagonist to inhibit transcriptional activity of a polypeptide wherein the polypeptide comprises a NURR subfamily amino acid sequence, such as one selected from the group consisting of SEQ ID NO:33, SEQ ID NO:64 and SEQ ID NO:91, wherein the polypeptide is a nuclear receptor. In a specific embodiment, the polypeptide is a steroid receptor. In a specific embodiment, the polypeptide is a hormone receptor. In a specific embodiment, the polypeptide is a vitamin receptor. In a specific embodiment the antagonist inhibits arthritis. In another specific embodiment, the antagonist inhibits joint inflammation.

In a further embodiment of the present invention, there is an antagonist to inhibit transcriptional activity of a polypeptide wherein the polypeptide comprises a NURR1 amino acid sequence of SEQ ID NO:33, and wherein the polypeptide is a nuclear receptor. In a specific embodiment the antagonist inhibits arthritis. In another specific embodiment, the antagonist inhibits joint inflammation.

In another embodiment, there is a method of screening for a compound that interferes with an interaction of a NURR subfamily polypeptide with a ligand comprised of introducing to a cell a test agent, wherein the cell comprises a marker sequence, wherein the expression of the marker sequence is regulated by said NURR subfamily member, and measuring the expression level of the marker sequence, wherein when the expression of the marker sequence is reduced following the introduction, the test agent is the compound that interferes with an interaction of a NURR subfamily polypeptide with a ligand. A specific embodiment is the compound identified by screening for a compound that interferes with an interaction of a NURR subfamily polypeptide with a ligand as a composition of matter. In a further specific embodiment, the NURR subfamily polypeptide is a sequence selected from the group consisting of SEQ ID NO:33, SEQ ID NO:64 and SEQ ID NO:91. In another specific embodiment, the NURR subfamily polypeptide is a sequence of SEQ ID NO.:33.

In another embodiment of the present invention, there is a method of identifying a compound for the treatment of an inflammatory immune disease comprising introducing to a cell a test agent, wherein the cell comprises a marker sequence, wherein the expression of the marker sequence is regulated by the NURR subfamily member, and measuring the expression level of the marker sequence, wherein when the expression of the marker sequence is reduced following introduction of the test agent, the test agent is the compound for treatment of inflammatory immune disease. In a specific embodiment, the inflammatory immune disease is in a joint.

In another embodiment of the present invention, there is a pharmacologically acceptable composition comprising the compound identified for the treatment of an inflammatory immune disease and a pharmaceutical carrier. In a specific embodiment, the compound for the treatment of an inflammatory disease is dispersed in a pharmaceutical carrier and administered in a therapeutically effective amount of the compound in the carrier to an individual having inflammatory immune disease.

In another embodiment of the present invention, there is an agonist of transcriptional activity of a polypeptide wherein the polypeptide comprises a NURR subfamily amino acid sequence selected from the group consisting of SEQ ID NO:33, SEQ ID NO:64 and SEQ ID NO:91, and wherein the polypeptide is a nuclear receptor. In a specific embodiment, the agonist is a steroid receptor. In a specific embodiment, the agonist is a hormone receptor. In a specific embodiment, the agonist is a vitamin receptor.

In a further embodiment of the present invention there is an agonist of transcriptional activity of a polypeptide wherein the polypeptide comprises a NURR1 amino acid sequence of SEQ ID NO:33 and wherein the polypeptide is a nuclear receptor.

In a specific embodiment of the present invention there is a method of treating an organism for an inflammatory immune disease comprising the step of reducing expression of a CRH receptor nucleic acid sequence. In a further specific embodiment, the reducing of CRH receptor expression comprises inhibiting synthesis of a nucleic acid sequence of SEQ ID NO: 104. In another specific embodiment of the present invention there is a method of treating an organism for an inflammatory immune disease comprising the step of reducing the level of a CRH receptor amino acid sequence. In a further specific embodiment, the reduction of the CRH receptor amino acid level comprises inhibiting amino acid synthesis, increasing a CRH receptor amino acid breakdown, or comprises administering therapeutically effective levels of antibodies to the CRH receptor polypeptide of a sequence comprising SEQ ID NO:124. In a specific embodiment, the inflammatory immune disease is selected from the group consisting of chronic inflammatory joint disease, arthritis, rheumatoid arthritis, ulcerative colitis and thyroiditis. In another specific embodiment the inflammatory joint disease is arthritis. In an additional specific embodiment the arthritis is selected from the group consisting of rheumatoid arthritis, psoriatic arthritis and sarcoid arthritis.

Other and further objects, features and advantages would be apparent and eventually more readily understood by reading the following specification and by reference to the company drawing forming a part thereof, or any examples of the presently preferred embodiments of the invention are given for the purpose of the disclosure.

### DESCRIPTION OF THE DRAWINGS

FIGS. 1A through 1D demonstrate that CRH MRNA expression in human synovial tissue and primary synoviocytes.

FIGS. 2A through 2B demonstrate that pro-inflammatory mediators activate transcription from the hCRH promoter in cultured synoviocytes.

FIGS. 3A through 3D show immunohistochemical staining for CRH receptors in inflamed synovium.

FIGS. 4A through 4B demonstrate northern analysis of NURR1 and NUR77 MRNA in RA synovium explants and cultured primary synoviocytes.

FIGS. 5A through 5D show immunohistochemical staining of synovial tissue and cultured synoviocytes with anti-NURR1 immune serum.

FIGS. 6A through 6E show effects of pro-inflammatory mediators, dexamethasone, indomethacin, and cycloheximide on NURR1 mRNA expression in synoviocytes.

FIG. 7 demonstrates electrophoretic mobility shift analysis (EMSA) of nuclear extracts from primary synoviocytes.

FIG. 8 illustrates modulation of locally produced CRH is a component of the cytokine network in human inflammatory arthritis. Pro-inflammatory mediators associated with inflammatory arthritis increase synovial CRH production. Synovial CRH induces the nuclear transcription factor NURR1 in CRH-receptor bearing endothelial and some mononuclear cells. NURR1 contributes to cytokine-mediated signaling and is a general mediator of an autocrine inflammatory cascade, which further serves to amplify the inflammatory response by increasing CRH expression. Dexamethasone (DEX) functions by inhibiting both cytokine and CRH-induced NURR1 mRNA expression.

FIGS. 9A and 9B demonstrate the binding of the human NURR1 promoter fused to a β-galactosidase reporter gene to oligonucleotides corresponding to the NFκB binding sequence.

FIG. 9D demonstrates the binding of human NURR1 promoter fused to a β-galatosidase reporter gene to oligonucleotides corresponding to the CREB binding sequence.

FIG. 9C shows β-galactosidase activity in primary RA synoviocytes and synovial tissue transfected with the reporter.

FIGS. 10A through 10D illustrate immunohistochemical staining of cultured synoviocytes with anti-NURR1 immune serum.

FIGS. 11A through 11F show immunohistochemical staining of synovial tissues to determine expression of CRH-R1 and CRH-R2 receptor subtypes.

FIGS. 12A and 12B show the immunolocalization of mast cell tryptase and CRH-R1 using a double antibody staining method.

FIGS. 13A through 13D characterize CRH receptor subtype mRNA expression in synovial tissue.

FIG. 14 illustrates NORI and NURR1 expression in primary RA synoviocytes by Northern analysis.

### DESCRIPTION OF THE INVENTION

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

As used herein the specification, “a” or “an” may mean one or more. As used herein in the claim(s), when used in conjunction with the word “comprising”, the words “a” or “an” may mean one or more than one. As used herein “another” may mean at least a second or more.

The term “agonist” as used herein is defined as a factor which promotes, facilitates or enhances the activity or function of another biological entity. In a specific embodiment, it is an agonist of transcriptional activity of a NURR subfamily polypeptide. In another specific embodiment, it is an agonist of a polypeptide encoding a NURR1 amino acid sequence, wherein the polypeptide is a nuclear receptor. The agonist may be an amino acid sequence, a nucleic acid sequence, a lipid, a sugar, a carbohydrate, or a combination thereof. In a specific embodiment, the agonist is associated with inflammation. Examples include, but are not limited to, IL1_{β}, TNF_{α}, IL-6 and PGE₂. Other terms for the same agents as used herein are mediators and cytokines.

The term “antagonist” as used herein is defined as a factor which interferes with, neutralizes or impedes the activity, function or effect of another biological entity. In a specific embodiment, the antagonist inhibits transcriptional activity of a NURR subfamily polypeptide. In another specific embodiment, it is an antagonist of a polypeptide encoding a NURR1 amino acid sequence, wherein the polypeptide is a nuclear receptor. The antagonist may be an amino acid sequence, a nucleic acid sequence, a lipid, a sugar, a synthetic chemical molecule, a hapten, a carbohydrate, or a combination thereof. The agent may partially or completely interfere with a NURR1 activity. In a specific embodiment, the antagonist ligand inhibits NURR1 transcriptional activity.

The term “anti-cytokine” as used herein is defined as a biological agent which interferes with the synthesis, activity or function of a cytokine. The biological agent may be an amino acid, a nucleic acid, a lipid, a sugar, a carbohydrate, or combination thereof. Interference with the cytokine may comprise a direct or indirect interaction. The anti-cytokine may be endogenous or synthetically derived.

The term “arthritis” as used herein is defined as inflammation of a joint. In specific embodiments, the joint is of a shoulder, knee, elbow, knuckle, finer, knee ankle, neck or hip. In another specific embodiment, multiple joints are affected.

The term “cytokine” as used herein is defined as a soluble substance produced by lymphoid or non-lymphoid cells which have the ability to cause the same effects on target cells as a lymphokine, including promoting inflammation. Lymphokine is herein defined as biologically active soluble factors released by lymphoblasts in response to antigen invasion. Examples of cytokines include, but are not limited to, IL1_{β}, TNF_{α}, IL-6 and PGE₂.

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

The term “inflammatory immune disease” as used herein is defined as a disease which affects the immune system of an organism, causing inflammation of particular regions of the body. In a specific embodiment, the regions of inflammation include the synovial fluid of the joints, the colon and the thyroid. The inflammation may be a primary symptom of the disease or may be indirectly related to the disease. In a specific embodiment, the inflammation may be a low level grade of inflammation such as with a degenerative form of arthritis including osteoarthritis. Examples of inflammatory immune diseases include arthritis, such as rheumatoid arthritis, psoriatic arthritis and sarcoid arthritis, osteoarthritis, ulcerative colitis and thyroiditis.

The term “interferes” as used herein is defined as retarding, slowing down, or impeding an action to prevent an undesirable result. The interference may be complete or may be partial.

The term “inhibits” as used herein is defined as blocking, retarding, or impeding an action to prevent an undesirable result. The inhibition may be complete or may be partial.

The term “ligand” as used herein is defined as a molecule that binds to another molecule. In a specific embodiment, a ligand that binds to a NURR subfamily member is preferred. One skilled in the are recognizes that a ligand includes the whole ligand, any part and any mutant thereof that remains capable of binding to a NURR subfamily member.

The term “NURR subfamily” as used herein is defined as a group of nuclearly located transcription factors that function as constitutively active transcription factors and are related to NURR1 (Nur-related factor I), wherein the relationship is structural and functional. In a specific embodiment, the subfamily is characterized by the ability of the different members to functionally complement one another. In another specific embodiment, the subfamily have identical sequence of the DNA binding domain, wherein the sequence identity is approximately 40%. In further another specific embodiment, the sequence identity is approximately 45%. In further another specific embodiment, the sequence identity is approximately 50%. In further another specific embodiment, the sequence identity is approximately 55%. In further another specific embodiment, the sequence identity is approximately 60%. In further another specific embodiment, the sequence identity is approximately 65%. In further another specific embodiment, the sequence identity is approximately 70%. In further another specific embodiment, the sequence identity is approximately 75%. In further another specific embodiment, the sequence identity is approximately 80 %. In further another specific embodiment, the sequence identity is approximately 85 %. In further another specific embodiment, the sequence identity is approximately 90 %. In further another specific embodiment, the sequence identity is approximately 95 %. In further another specific embodiment, the sequence identity is approximately 99%. The family includes NURR1, NOR1 (neuron derived orphan receptor) and NURR77. See Maruyama et al., 1995 herein incorporated by reference, for a discussion of the subfamily and Table 1 there in regarding alternative names for each member. A skilled artisan recognizes that the group may also be referred to as the NGFI-B subfamily of a nuclear receptor superfamily. Characteristics may include a central DNA binding domain comprising two highly conserved zinc finger motifs (Berg, 1989; Klug and Schwabe, 1995), a ligand-binding domain comprising 8-9 heptad repeats of hydrophobic amino acids in the carboxyl terminus, and a variable amino-terminal region.

The term “orphan receptor” as used herein refers to molecules that are structurally-related to known receptors, wherein the identity of the ligand and physiological function is unknown.

The term “polypeptide” as used herein is defined as a molecule which comprises more than one amino acid subunits. The polypeptide may be an entire protein or it may be a fragment of a protein, such as a peptide or oligopeptide. The polypeptide may also comprise alterations to the amino acid subunits, such as methylation or acetylation. In a specific embodiment, the polypeptide has a nuclear localization sequence or sequences.

The term “receptor” as used herein is defined as a biological entity which associates with or is a NURR subfamily amino acid sequence. The receptor may be an amino acid sequence, a nucleic acid sequence, a lipid, a sugar, a carbohydrate or combination thereof. In a specific embodiment, the receptor is an amino acid sequence. The receptor may be located in a membrane, in the nucleus, or in the cytoplasm.

The term “therapeutically effective” as used herein is defined as the amount of a compound required to improve some symptom associated with a disease. For example, in the treatment of an inflammatory immune disease such as arthritis, a compound which decreases, prevents, delays or arrests any symptom of the disease would be therapeutically effective. A therapeutically effective amount of a compound is not required to cure a disease but will provide a treatment for a disease. A compound is the to be administered in a therapeutically effective amount if the amount administered is physiologically significant. A compound is physiologically significant if its presence results in technical change in the physiology of a recipient organism.

The term “transcribed” as used herein refers to the generation of a ribonucleic acid from a deoxyribonucleic acid template.

The term “treatment” as used herein is defined as the management of a patient through medical or surgical means. The treatment improves or alleviates at least one symptom of a medical condition or disease and is not required to provide a cure.

The term “vascular disease” as used herein is defined as any disease in which blood vessels, including arteries, veins and capillaries, are restricted in diameter. The vascular disease may involve changes in vascular permeability or vasodilation, such as angiogenesis. The restricted blood vessels may be a primary symptom of a disease or medical condition, such as heart disease.

In an embodiment of the invention, there is a method of treating an organism for an inflammatory immune disease comprising the step of reducing the level of a polypeptide comprising a NURR subfamily amino acid sequence of SEQ ID NO:2. In a specific embodiment, reduction of the levels comprises administering therapeutically effective levels of an antagonist to the polypeptide. In another specific embodiment, the antagonist is selected from the group consisting of an amino acid, a nucleic acid, a lipid, an organic synthetic molecule, a hapten, a sugar, a carbohydrate, or a combination thereof. In a further specific embodiment the antagonist is an amino acid.

In another embodiment, of the present invention reduction of the polypeptide levels comprises inhibiting a NURR subfamily amino acid synthesis, increasing a NURR subfamily amino acid breakdown, or comprises administering therapeutically effective levels of antibodies to the NURR subfamily polypeptide. In a specific embodiment, the inflammatory immune disease is selected from the group consisting of chronic inflammatory joint disease, arthritis, rheumatoid arthritis, ulcerative colitis and thyroiditis. In another specific embodiment the inflammatory joint disease is arthritis. In an additional specific embodiment the arthritis is selected from the group consisting of rheumatoid arthritis, psoriatic arthritis and sarcoid arthritis. In another embodiment, there is a method for treating an inflammatory immune disease comprising the step of administering an anti-cytokine. In a specific embodiment, the anti-cytokine interferes with IL1_{β}, TNF_{α}, IL-6 or PGE₂. In a specific embodiment, the anti-cytokine is a glucocorticoid. In an additional embodiment, the reduction of the NURR subfamily polypeptide levels comprises reducing amino acid or ribonucleic acid levels of corticotropin releasing hormone, pro-opiomelanocorticotropin, collagenase (MMP-1), serum amyloid A, and PGE₂.

In an additional embodiment of the present invention, there is a method of treating an organism for an inflammatory immune disease comprising the steps of reducing levels of a NURR subfamily ribonucleic acid sequence transcribed from a NURR subfamily, such as a NURR1 nucleic acid sequence of SEQ ID NO: 1. In a specific embodiment, the reduction comprises inhibiting a NURR subfamily nucleic acid synthesis, administering therapeutically effective levels of an antisense sequence of the NURR subfamily nucleic acid sequence, or administering therapeutically effective levels of an anti-cytokine. In another specific embodiment the anti-cytokine interferes with a cytokine selected from the group consisting of IL1_{β}, TNF_{α}, IL-6 and PGE₂. In a specific embodiment, the anti-cytokine is a glucocorticoid. In an additional embodiment, the reduction of a NURR subfamily nucleic acid sequence levels comprises reducing amino acid or ribonucleic acid levels of corticotropin releasing hormone, pro-opiomelanocorticotropin, collagenase (MMP-1), and serum amyloid A. In a specific embodiment, the inflammatory immune disease is selected from the group consisting chronic inflammatory joint disease, arthritis, rheumatoid arthritis, ulcerative colitis and thyroiditis. In another specific embodiment the inflammatory immune disease is arthritis. In an additional specific embodiment the arthritis is selected from the group consisting of rheumatoid arthritis, psoriatic arthritis and sarcoid arthritis.

In another embodiment, of the present invention there is a method of treating an organism for an inflammatory immune disease comprising the step of interfering with binding of a polypeptide comprising a NURR subfamily, such as an amino acid sequence of SEQ ID NO:33, to a nucleic acid. In a specific embodiment, the interference comprises administering therapeutically effective levels of an antagonist to a NURR subfamily polypeptide, administering therapeutically effective levels of antibodies to a NURR subfamily polypeptide, or increasing to a therapeutically effective amount the levels of a receptor or nucleic acid which binds a NURR subfamily member. In another specific embodiment the antagonist is selected from the group consisting of an amino acid, a nucleic acid, a lipid, a sugar, a carbohydrate, or a combination thereof. In a further specific embodiment the antagonist is an amino acid. In a specific embodiment, the inflammatory immune disease is selected from the group consisting chronic inflammatory joint disease, arthritis, rheumatoid arthritis, ulcerative colitis and thyroiditis. In another specific embodiment the inflammatory immune disease is arthritis. In an additional specific embodiment the arthritis is selected from the group consisting of rheumatoid arthritis, psoriatic arthritis and sarcoid arthritis.

In an additional embodiment of the present invention there is a method of treating an organism for a vascular disease comprising the step of administering to the organism a therapeutically effective amount of a nucleic acid sequence of a NURR subfamily member. In a specific embodiment, the administration comprises a vector. In a further specific embodiment the vector is a nucleic acid, an amino acid, a lipid, a liposome, a sugar, a carbohydrate, or a combination thereof. In another specific embodiment the nucleic acid vector is an adenovirus, an adeno-associated virus, or a retrovirus.

In a specific embodiment, there is a method of treating an organism for an inflammatory immune disease comprising the step of reducing levels of a ribonucleic acid transcribed from a NURR1 nucleic acid sequence of SEQ ID NO: 1, wherein the NURR1 nucleic acid sequence encodes a vasodilator. In a specific embodiment, the reduction of the NURR1 ribonucleic acid levels comprises administering therapeutically effective levels of an antisense sequence of the NURR1 nucleic acid sequence. In an additional specific embodiment the nucleic acid sequence comprises a vector. In another specific embodiment the vector is selected from the group consisting of a nucleic acid, an amino acid, a lipid, a liposome, a sugar, a carbohydrate, and a combination thereof. In a further specific embodiment the nucleic acid vector is an adenovirus, an adeno-associated virus and a retrovirus.

In another embodiment, of the present invention there is a method of treating an organism for an inflammatory immune disease comprising the step of reducing levels of a polypeptide comprising a NURR subfamily amino acid sequence, wherein the NURR subfamily polypeptide acts as a vasodilator. In a specific embodiment, there is a method of treating an organism for a vascular disease comprising the step of administering to the organism therapeutically effective levels of a polypeptide comprising a NURR subfamily amino acid sequence of SEQ ID NO:33. In an additional embodiment, the administration of an amino acid sequence comprises a protein transduction domain. In a further specific embodiment the protein transduction domain is the HIV TAT protein transduction domain.

In another embodiment, of the present invention there is a method of preventing an inflammatory immune disease in an organism comprising the step of reducing levels of a ribonucleic acid transcribed from a NURR subfamily nucleic acid sequence, such as a sequence comprising SEQ ID NO:1.

In an additional embodiment of the present invention there is a method of preventing an inflammatory immune disease in an organism comprising the step of reducing the level of a polypeptide comprising a NURR subfamily amino acid sequence, such as a sequence comprising SEQ ID NO:33.

In an additional embodiment of the present invention there is an antagonist of a polypeptide wherein the polypeptide comprises a NURR subfamily amino acid sequence, such as a sequence comprising SEQ ID NO:33 and wherein the polypeptide is a nuclear receptor. In a specific embodiment, the antagonist is an amino acid. In other specific embodiments the polypeptide is a steroid receptor, a hormone receptor or a vitamin receptor.

In a further embodiment of the present invention there is an agonist of a polypeptide wherein the polypeptide comprises a NURR subfamily amino acid sequence such as a NURR1 sequence comprising SEQ ID NO:33, and wherein the polypeptide is a nuclear receptor. In a specific embodiment, the agonist is an amino acid. In other specific embodiments the polypeptide is a steroid receptor, a hormone receptor or a vitamin receptor.

In an embodiment of the present invention, there is a compound for the treatment of an inflammatory immune disease in an organism wherein the compound is an antagonist of a polypeptide comprising a NURR subfamily amino acid sequence, such as a sequence comprising SEQ ID NO:33 and wherein the polypeptide is a nuclear receptor. In a specific embodiment, the inflammatory immune disease is selected from the group consisting of a chronic inflammatory joint disease, arthritis, rheumatoid arthritis, ulcerative colitis and thyroiditis. In another specific embodiment the inflammatory immune disease is arthritis.

In an additional embodiment of the present invention, there is a compound for the treatment of an inflammatory immune disease in an organism wherein the compound is an agonist of a polypeptide comprising a NURR subfamily amino acid sequence, such as a NURR subfamily amino acid sequence comprising SEQ ID NO:33, and wherein the polypeptide is a nuclear receptor. In a specific embodiment, the inflammatory immune disease is selected from the group consisting of a chronic inflammatory joint disease, arthritis, rheumatoid arthritis, ulcerative colitis and thyroiditis. In another specific embodiment the inflammatory immune disease is arthritis.

In a specific embodiment, of the present invention there is a method of treating an organism for an inflammatory immune disease comprising the step of reducing expression of a CRH receptor nucleic acid sequence. In a further specific embodiment, the reducing of CRH receptor expression comprises inhibiting synthesis of a nucleic acid sequence of SEQ ID NO: 104. In another specific embodiment of the present invention there is a method of treating an organism for an inflammatory immune disease comprising the step of reducing the level of a CRH receptor amino acid sequence. In a further specific embodiment, the reduction of the CRH receptor amino acid level comprises inhibiting amino acid synthesis, increasing a CRH receptor amino acid breakdown, or comprises administering therapeutically effective levels of antibodies to the CRH receptor polypeptide of a sequence comprising SEQ ID NO: 124. In a specific embodiment, the inflammatory immune disease is selected from the group consisting of chronic inflammatory joint disease, arthritis, rheumatoid arthritis, ulcerative colitis and thyroiditis. In another specific embodiment the inflammatory joint disease is arthritis. In an additional specific embodiment the arthritis is selected from the group consisting of rheumatoid arthritis, psoriatic arthritis and sarcoid arthritis.

It is an object of the present invention to relate methods of treatment, methods of prevention, antagonists, agonists and compounds to all members of the NURR subfamily given the striking structurally and genetically related redundancy of the nuclear receptor superfamily members including NURR1, NOR1 and NUR77 as discussed above. One skilled in the art recognizes that within the scope of the invention a NURR1 sequence is utilized. Examples of nucleic acid NURR1 sequence followed by the Genbank accession number include SEQ ID NO:1 (AB017586), SEQ ID NO: 2 (NT005151), SEQ ID NO:3 (AJ278700), SEQ ID NO:4 (NM013613), SEQ ID NO:5 (NM006186), SEQ ID NO:6 (BB539587), SEQ ID NO:7 (BB536225), SEQ ID NO:8 (BB432168), SEQ ID NO:9 (BB424269), SEQ ID NO:10 (BB345745), SEQ ID NO:ll (BB322941), SEQ ID NO:12 (BB023391), SEQ ID N013 (BB023355), SEQ ID NO:14 (AB019433), SEQ ID NO:15 (XM002441), SEQ ID NO:16 (AV3566519), SEQ ID NO:17 (AV356512), SEQ ID NO:18 (AV382234), SEQ ID NO:19 (AV368035), SEQ ID NO:20 (AV352127), SEQ ID NO:21 (AV341553), SEQ ID NO:22 (AV245724), SEQ ID NO:23 (AV221665), SEQ ID NO:24 (A1B014889), SEQ ID NO:25 (U72345), SEQ ID NO:26 (U86783), SEQ ID NO:27 (U67738), SEQ ID NO:28 (U93471), SEQ ID NO:29 (U93429), SEQ ID NO:30 (S53744), SEQ ID NO:31 (R35928), and SEQ ID NO:32 (R25908). Examples of amino acid NURR1 sequence include SEQ ID NO:33 (548390), SEQ ID NO:34 (XP002441), SEQ ID NO:35 (CAC27783), SEQ ID NO:36 (A46225), SEQ ID NO:37 (NP038641), SEQ ID NO:38 (NP006177), SEQ ID NO:39 (BAA77328), SEQ ID NO:40 (BAA75666), SEQ ID NO:41 (Q07917), SEQ ID NO:42 (P43354), SEQ ID NO:43 (Q04913), SEQ ID NO:44 (AAB68748), SEQ ID NO:45 (AAB68706), and SEQ ID NO:46 (AAB25138). One skilled in the art recognizes that within the scope of the invention a NOR-1 sequence is utilized. Examples of nucleic acid NOR-1 sequence include SEQ ID NO:47 (1651190), SEQ ID NO:48 (D38530). SEQ ID NO:49 (AF050223), SEQ ID NO:50 (X75871), SEQ ID NO:51 (L2781), SEQ ID NO:52 (BG235965), SEQ ID NO:53 (BE656711), SEQ ID NO:54 (BE188095), SEQ ID NO:55 (BE187931, SEQ ID NO:56 (AJ011768), SEQ ID NO:57 (E14965), SEQ ID NO:58 (AJ011767), SEQ ID NO:59 (D85244), SEQ ID NO:60 (D85243), SEQ ID NO:61 (D85242), SEQ ID NO:62 (D85241), and SEQ ID NO:63 (NM015743). Examples of amino acid NOR-1 sequence include SEQ ID NO:64 (7441771), SEQ ID NO:65 (Q92570), SEQ ID NO:66 (BAA11419), SEQ ID NO:67 (JC2493), SEQ ID NO:68 (NP056558), SEQ ID NO:69 (P51179), SEQ ID NO:70 (CAA09764), SEQ ID NO:71 (CAA09763), SEQ ID NO:72 (BAA31221), SEQ ID NO:73 (BAA28608), SEQ ID NO:74 (BAA07535), and SEQ ID NO:75 (AAA32685). One skilled in the art recognizes that within the scope of the invention a NUR77 sequence is utilized. Examples of nucleic acid NUR77 sequence include SEQ ID NO:76 (1339917), SEQ ID NO:76 (12662548), SEQ ID NO:77 (13F937382), SEQ ID NO:78 (NM006981), SEQ ID NO:79 (AR085655), SEQ ID NO:80 (AR085654), SEQ ID NO:81 (AR085653), SEQ ID NO:82 (AR085654), SEQ ID NO:83 (AR085652), SEQ ID NO:84 (BE198460), SEQ ID NO:85 (BE047656), SEQ ID NO:86 (BE047651), SEQ ID NO:87 (AW988827), SEQ ID NO:88 (AA461422), SEQ ID NO:89 (D49728), and SEQ ID NO:90 (S77154). Examples of amino acid NUR77 sequence include SEQ ID NO:76 (127819), SEQ ID NO:91 (128911), SEQ ID NO:92 (P22829), SEQ ID NO:93 (NP034574), SEQ ID NO:94 (AAB33999), SEQ ID NO:95 (NP008912), SEQ ID NO:96 (AAA42058), and SEQ ID NO:97 (A37251). A skilled artisan would know how to retrieve sequences from the National Center for Biotechnology Genbank database or commercially available databases such as the genetic database by Celera.

In the present invention, the methods are used for either treating an inflammatory immune disease, such as arthritis, or prevention of such a disease. Examples of use in the treatment would be for the improvement of the inflammatory immune disease after its onset or in helping alleviate its symptoms. The inflammatory immune disease is considered to be improved if at least one symptom is alleviated, wherein alleviation may be partial or complete. An example of use for the treatment for prevention would be the use prior to the onset of arthritis to prevent inflammation of synovium or synovial fluid, and thus prevent or delay the onset of arthritis.

One specific embodiment of the present invention is a method of treating arthritis comprising the step of lowering a NURR subfamily member nucleic acid levels. In a specific embodiment, NURR1 nucleic acid is lowered. Another specific embodiment of the present invention includes the method of treating arthritis comprising the step of lowering a NURR subfamily member amino acid levels. In a specific embodiment, NURR1 amino acid levels are lowered. One skilled in the art recognizes that there are a variety of ways to lower NURR1 nucleic acid or amino acid levels.

Screening Assays—Amino Acid Antagonists

In a specific embodiment of the present invention there is a method of administering an antagonist to a NURR subfamily amino acid sequence. In another embodiment, thers is a method of administering an antagonist to a CRH receptor amino acid sequence. A skilled artisan recognizes that the antagonist in one embodiment interferes with NURR transcriptional activity by binding to the NURR subfamily member. In another embodiment, the action of the antagonist results in reduced expression of a NURR subfamily member. A skilled artisan is aware that standard methods are utilized to screen for compounds which inhibits or acts as an antagonist to a NURR subfamily amino acid sequence or a CRH receptor amino acid sequence. Compound banks or oligopeptide libraries are screened In a specific embodiment, by methods well known in the art. The antagonist may be an amino acid, nucleic acid, lipid, liposome, carbohydrate, sugar or combination thereof. In a preferred embodiment, the antagonist is an amino acid.

Another embodiment of the present invention includes lowering a NURR subfamily member levels by administering antibodies to a NURR subfamily member to sequester NURR subfamily member amino acid sequence from available pools. Another further embodiment of the present invention includes lowering a CRH receptor levels by administering antibodies to a CRH receptor to sequester CRH receptor amino acid sequence from available pools. NURR subfamily amino acid sequence and CRH receptor amino acid sequence for antibody induction do not require biological activity; however, the protein fragment, or oligopeptide must be antigenic. Peptides used to induce specific antibodies may have an amino acid sequence consisting of at least about five amino acids, preferably at least about 10. They should mimic a portion of the amino acid sequence of the natural protein and may contain the entire amino acid sequence of a small, naturally occurring molecule. Procedures well known in the art can be used for the production of antibodies to a NURR subfamily member amino acid sequence or for the production of antibodies to a CRH receptor amino acid sequence.

For the production of antibodies, various hosts including goats, rabbits, rats, mice, etc., may be immunized by injection with NURR subfamily member protein or CRH receptor protein or any portion, fragment, or oligopeptide thereof which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants. Monoclonal antibodies to a NURR subfamily member or CRH receptor is prepared using any technique which provides for the production of antibody molecules by continuous cell lines in culture. Derivatives of antibodies and fragments that retain antigen binding sites are also included in the present invention.

In a specific embodiment, levels of NURR subfamily member amino acid sequence or CRH receptor amino acid sequence are reduced by inhibiting a NURR subfamily member amino acid synthesis or a CRH receptor amino acid synthesis. This includes not only prevention or cessation of translation of a NURR1 sequence or a CRH receptor sequence but also includes posttranslational processing and transport to proper subcellular localization.

In another embodiment, the levels of a NURR subfamily amino acid sequence or CRH receptor amino acid sequence are reduced by increasing a NURR subfamily amino acid breakdown or CRH receptor breakdown, respectively. This includes modification to the NURR subfamily amino acid sequence or CRH receptor amino acid sequence which targets the amino acid sequence for degradation, such as ubiquitination.

In a further embodiment the NURR subfamily amino acid levels are decreased by decreasing CRH nucleic acid or amino acid levels. As demonstrated in the Examples, CRH induces expression of NURR1. Therefore, reducing levels by analogous means described herein for NURR1 suppression In a specific embodiment, also reduces NURR1 amino acid levels. In an analogous method, other NURR subfamily members are reduced by decreasing CRH levels.

In a preferred embodiment, reducing the amino acid levels of a NURR subfamily member amino acid sequence further comprises decreasing nucleic acid or amino acid sequences of collagenase or serum amyloid A, two examples of genes with NURR1 consensus binding sites in their regulatory region which have been implicated in RA inflammation mechanisms and joint destruction. Other nucleic acid sequences regulated by NURR1 and related to inflammation associated with an immune disease are within the scope of the invention. Collagenase nucleic acid sequences are herein represented by SEQ ID NO:141 (13639671) and amino acid sequences are herein represented by SEQ ID NO:142(13639672). Serum amyloid A nucleic acid sequence is herein represented by SEQ ID NO:98 (178868) and amino acid sequence is SEQ ID NO:99 (13540475). Other examples of genes which contain consensus binding sites in their regulatory regions are corticotropin releasing hormone (CRH) and pro-opiomelanocorticotropin (POMC). CRH nucleic acid comprises SEQ ID NO:100 (12803538) and amino acid sequence comprises SEQ ID NO:101 (AAH02599). POMC nucleic acid sequence is represented by SEQ ID NO:102 (11429780) and amino acid sequence is represented by SEQ ID NO:103 (13637253).

In a preferred embodiment, reducing the amino acid levels of a NURR subfamily member amino acid sequence further comprises decreasing nucleic acid or amino acid sequences of CRH receptor subtype R1. One skilled in the art recognizes that within the scope of the invention a CRH-R1 sequence is utilized. Examples of nucleic acid CRH-R1 sequence include SEQ ID NO:104 (5815472), SEQ ID NO:105 (accession no. NM030999), SEQ ID NO:106 (accession no. AB055434) SEQ ID NO:107 (accession no. NM007762), SEQ ID NO:108 (accession no. NM004382), SEQ ID NO:109 (accession no. BB523399), SEQ ID NO:110 (accession no. BB520290), SEQ ID NO:111 (accession no. BB517689), SEQ ID NO:112 (accession no. BB477388), SEQ ID NO:113 (accession no. BB475687), SEQ ID NO:114 (accession no. AF180301), SEQ ID NO:115 (accession no. BB239486), SEQ ID NO:116 (accession no. BB237761), SEQ ID NO:117 (accession no. BB169114), SEQ ID NO:118 (accession no. AV332164), SEQ ID NO:119 (accession no. AI561856), SEQ ID NO: 120 (accession no. U19939), SEQ ID NO: 121 (accession no. AF077185), SEQ ID NO:122 (accession no. AA543299), and SEQ ID NO:123 (accession no. BB009745). Examples of CRH-RI amino acid sequence include SEQ ID NO:124 (5815473), SEQ ID NO:125 (accession no. 062772), 042602), SEQ ID NO:126 (accession no. Q90812), SEQ ID NO:127 (accession no. P35353), SEQ ID NO:128 (accession no. P35347), SEQ ID NO:129 (accession no. P34998), SEQ ID NO:130 (accession no. NP112261), SEQ ID NO:131 (accession no. BAB21864), SEQ ID NO:132 (accession no. 138879), SEQ ID NO:133 (accession no. A48260), SEQ ID NO:134 (accession no. S39535), SEQ ID NO:135 (accession no. NP0031788), SEQ ID NO:136 (accession no. AAD52688), SEQ ID NO:137 (accession no. AAC52243), SEQ ID NO:138 (accession no. AAC50073), SEQ ID NO:139 (accession no. AAC27320) and SEQ ID NO:140 (accession no. NP004373).

In an additional embodiment, there is a treatment for an inflammatory immune disease which further comprises administration of an anti-cytokine. The anti-cytokine interferes with a cytokine, either directly or indirectly. In a specific embodiment, the cytokine which is the target of interference is IL1_{β}, TNF_{α}, IL-6 or PGE₂.

In an embodiment of the present invention there is a method to treat an organism for an inflammatory immune disease comprising reducing levels of a NURR subfamily nucleic acid sequence or of a CRH receptor nucleic acid sequence. The reduction of nucleic acid sequence levels of a NURR subfamily or CRH receptor can be by standard methods in the art. This includes reducing levels of a functional nucleic acid sequence and may comprise affecting posttranscriptional processing, application of 5′ mRNA cap, splicing and polyadenylation. In a specific embodiment, the NURR subfamily nucleic acid sequences no longer localize to proper subcellular locales. In another embodiment, the levels of a NURR subfamily nucleic acid sequence or CRH receptor nucleic acid sequence are reduced by affecting an upstream factor, such as a transcription factor which regulates expression of a NURR subfamily nucleic acid sequence. In a further embodiment the levels of a CRH receptor nucleic acid sequence are reduced by affecting an upstream factor, such as a transcription factor which regulates expression of a CRH receptor nucleic acid sequence.

Screening Assays—Nucleic Acid Antagonists

In an embodiment of the present invention there is a method to reduce nucleic acid levels of a NURR subfamily member. An example presented herein provides candidate substance screening methods that are based upon whole cell assays, in vivo analysis or transformed or immortal cell lines in which a reporter gene is employed to confer on its recombinant hosts a readily detectable phenotype that emerges only under conditions where a NURR subfamily member would have reduced levels of expression. As an example, reporter genes encode a polypeptide not otherwise produced by the host cell that is detectable by analysis, e.g., by chromogenic, fluorometric, radioisotopic or spectrophotometric analysis. In a specific embodiment, the NURR subfamily nucleic acid sequence which encodes the amino acid sequence has been replaced with β-galactosidase.

Another example of a screening assay of the present invention is presented herein. NURR subfamily member expressing cells are grown in microtiter wells, followed by addition of serial molar proportions of a candidate to a series of wells, and determination of the signal level after an incubation period that is sufficient to demonstrate expression in controls incubated solely with the vehicle which was used to resuspend or dissolve the compound. The wells containing varying proportions of candidate are then evaluated for signal activation. Candidates that demonstrate dose related reduction of reporter gene transcription or expression are then selected for further evaluation as clinical therapeutic agents.

In an alternative embodiment there is a method for reducing a NURR subfamily member nucleic acid levels by transfecting cells with antisense sequences of a sequence of a NURR subfamily member such as SEQ ID NO:1. Delivery systems for tranfection of nucleic acids into cells may utilize either viral or non-viral methods. A targeted system for non-viral forms of DNA or RNA requires four components: 1) the DNA or RNA of interest; 2) a moiety that recognizes and binds to a cell surface receptor or antigen; 3) a DNA binding moiety; and 4) a lytic moiety that enables the transport of the complex from the cell surface to the cytoplasm. Further, liposomes and cationic lipids can be used to deliver the therapeutic gene combinations to achieve the same effect. Potential viral vectors include expression vectors derived from viruses such as adenovirus, vaccinia virus, herpes virus, and bovine papilloma virus. In addition, episomal vectors may be employed. Other DNA vectors and transporter systems are known in the art.

One skilled in the art recognizes that expression vectors derived from retroviruses, adenovirus, herpes or vaccinia viruses, or from various bacterial plasmids, may be used for delivery of nucleotides sequences to a targeted organ, tissue or cell population. Methods which are well known to those skilled in the art can be used to construct recombinant vectors which will express antisense nucleotides of the gene encoding a NURR subfamily member or a CRH receptor. The genes can be turned off by transfecting a cell or tissue with expression vectors which express high levels of a desired gene-encoding fragment. Such constructs can flood cells with untranslatable sense or antisense sequences. Even in the absence of integration into the DNA, such vectors may continue to transcribe RNA molecules until all copies are disabled by endogenous nucleases. Transient expression may last for a month or more with a non-replicating vector and even longer if appropriate replication elements are a part of the vector system.

Furthermore, the skilled artisan recognizes that modifications of gene expression can be obtained by designing antisense molecules to the control regions of a NURR subfamily member nucleic acid sequence, i.e. the promoters, enhancers, and introns. Oligonucleotides derived from the transcription initiation site, e.g. between −10 and +10 regions of the leader sequence, are preferred. The antisense molecules may also be designed to block translation of mRNA by preventing the transcript from binding to ribosomes. Similarly, inhibition can be achieved by using “triple helix” base-pairing methodology. Triple helix pairing compromises the ability of the double helix to open sufficiently for the binding of polymerases, transcription factors, or regulatory molecules.

Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of the invention are engineered hammerhead motif ribozyme molecules that can specifically and efficiently catalyze the endonucleolytic cleavage of sequences encoding a NURR subfamily member. In another embodiment, the ribozyme is a Tetrahymena-type ribozyme.

Antisense molecules and ribozymes of the invention may be prepared by any method known in the art for the synthesis of RNA molecules, including techniques for chemically synthesizing oligonucleotides. Alternatively, RNA molecules may be generated by in vitro and in vivo transcription of DNA sequences encoding a NURR subfamily member or a NURR subfamily member receptor. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

In a specific embodiment, the transfection of nucleic acid is facilitated by a transport protein, as described in Subramanian et al. (1999). Briefly, a peptide M9 is chemically bound to a cationic peptide as a carrier molecule. The cationic complex binds the negatively charged nucleic acid of interest, followed by binding of M9 to a nuclear transport protein, such as transportin.

An embodiment of the present invention is to decrease a NURR subfamily member levels by increasing the synthesis of a NURR subfamily member receptor, which binds to a free NURR subfamily member.

In an embodiment of the present invention there is a method to decrease a NURR subfamily member nucleic acid levels comprising administering therapeutically effective levels of an anti-cytokine. In a specific embodiment, the anti-cytokine interferes with, for example, IL1_{β}, TNF_{α}, IL-6 or PGE₂.

In an additional embodiment of the present invention there is a decrease in NURR subfamily member nucleic acid levels, such as NURR1, which further comprises decreasing nucleic acid levels of a nucleic acid sequence associated with inflammatory immune disease, such as collagenase, serum amyloid A, CRH or POMC. In a specific embodiment, the decrease in a NURR subfamily member nucleic acid levels decreases a nucleic acid sequence which has a NURR subfamily member binding site in a regulatory region.

One specific embodiment of the present invention is a method for the administration of a factor which binds to a NURR subfamily member amino acid sequence to block, interfere with or modulate its biological or immunological activity, thereby rendering it unable to produce action on a NURR subfamily member receptor. The antagonist may include proteins, peptides, soluble receptors, nucleic acids, carbohydrates, lipids, sugars or other molecules which bind to a NURR subfamily member receptor.

One embodiment of the present invention is a method to administer antibodies to a NURR subfamily member, thereby preventing it from binding to a NURR subfamily member receptor.

In a specific embodiment, there is a method of interfering with a NURR subfamily member binding to a receptor by increasing to a therapeutically effective amount the level of a NURR subfamily member receptor. Such a method could be achieved by gene therapies known in the art and discussed herein or by administering a NURR subfamily member receptor amino acid level by methods standard in the art and also discussed herein.

One embodiment of the present invention is a method to administer compounds which affect a NURR subfamily member receptor structure. Such compounds may include but are not limited to proteins, peptides, nucleic acids, carbohydrates, or other molecules which upon binding alter a NURR subfamily member receptor structure, thereby rendering it ineffectual in its activity.

One embodiment of the present invention is a method to administer a compound or compounds which affect a NURR subfamily member receptor function. Such compounds may include but are not limited to proteins, nucleic acids, carbohydrates, or other molecules which upon binding inhibit or suppress function of a NURR subfamily member receptor.

In a specific embodiment, there is a method of treating an organism with a vascular disease comprising administering therapeutically effective levels to an organism an amino acid or nucleic acid sequence of a NURR subfamily member.

In a specific embodiment, there is a method of treating an organism for an inflammatory immune disease comprising the step of reducing nucleic acid levels of a NURR subfamily member, wherein the NURR subfamily member acts as a vasodilator. In rheumatoid arthritis the blood vessel plays an important role and the association of NURR1 and CRH in the vasculature, shown in the Examples, is thought to be an initial trigger for the disease. Therefore, it would be an obvious and beneficial strategy for treatment to reduce levels of a NURR subfamily member and CRH at an early stage of the disease process. In a specific embodiment, antisense NURR subfamily member, such as NURR1 is administered to the organism to promote vasoconstriction.

In another embodiment, the treatment for an inflammatory immune disease in an organism comprises the step of reducing amino acid levels of a NURR subfamily member, wherein a NURR subfamily member is a vasodilator.

In another embodiment, there is a method of preventing an inflammatory immune disease in an organism comprising the step of reducing levels of a NURR subfamily member nucleic acid or amino acid sequence. The administration can be to organisms which show no signs of the onset of the inflammatory immune disease or have early signs of the disease. In a preferred embodiment, the organism is susceptible to the inflammatory immune disease or shows a genetic predisposition to having the disease.

In a specific embodiment, the methods and treatments described herein are used in conjunction with other anti-inflammatory therapies, including anti-cytokine treatments known in the art.

In a preferred embodiment, the organism described herein to be treated or subject to preventative methods is a human.

The methods and treatments described herein are directed to an inflammatory disease. In a specific embodiment, the disease is systemic, and therapies would be administered to patients systemically. However, in an alternative embodiment the therapies may be administered by direct application, such as by injection, to an inflamed body region such as a joint.

Dosage and Formulation

The compounds (active ingredients) of this invention can be formulated and administered to treat an inflammatory immune disease by any means that produces contact of the active ingredient with the agent's site of action in the body of a vertebrate. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

The active ingredient can be administered orally in solid dosage forms such as capsules, tablets and powders, or in liquid dosage forms such as elixirs, syrups, emulsions and suspensions. The active ingredient can also be formulated for administration parenterally by injection, rapid infusion, nasopharyngeal absorption or dermoabsorption. The agent may be administered intramuscularly, intravenously, subcutaneously, transdermally or as a suppository. In administering a compound, the compound may be given systematically. For compounds which require avoidance of systemic effects, a preferred embodiment is intrathecal administration. In a preferred embodiment, of the invention the compound is administered interarticularly for the treatment of arthritis.

Gelatin capsules contain the active ingredient and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows. Pharmacological ranges for the active ingredients can be determined by the skilled artisan using methods well known in the art. Example ranges for active ingredients are as follows: folate ranges between 400 micrograms and 4 milligrams/day; methionine ranges between 250 mg (total) and as high as 100 mg/kg/day daily, up to 2-3 g; choline ranges between 100 mg and 2 grams; Vitamin B12 at approximately 100 micrograms orally or 1 mg intramuscularly per month; betaine ranges up to 6 grams per day; zinc ranges between 25 and 50 mg; and sodium phenylbutyrate ranges up to 20 grams per day.

Capsules: Capsules are prepared by filling standard two-piece hard gelatin capsulates each with powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the active ingredient. The capsules are then washed and dried.

Tablets: Tablets are prepared by conventional procedures so that the dosage unit contains the suggested amount of active ingredient, 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 milliliters contains the suggested amount of finely divided active ingredient, 200 milligrams of sodium carboxymethyl cellulose, 5 milligrams of sodium benzoate, 1.0 grams of sorbitol solution U.S.P. and 0.025 milliliters of vanillin.

Accordingly, the pharmaceutical composition of the present invention may be delivered via various routes and to various sites in an animal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The composition of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

In a specific embodiment, a drug may be transported to a target by utilizing carbonic anhydrase inhibitor (CAI) which contains a polar group such as a carboxyl group, as described in Kehayova et al., 1999. The carboxyl group renders the composition dissolvable in water, however, upon exposure to light the bond linking the CAI to the carboxyl mask breaks, allowing the remaining portion to be soluble in a hydrophobic environment.

In certain embodiments, the use of lipid formulations and/or nanocapsules is contemplated for the introduction of an antagonist, an agonist, a polypeptide comprising a NURR1 amino acid sequence of SEQ ID NO:33, a nucleic acid comprising a NURR1 nucleic acid sequence of SEQ ID NO:1, a polypeptide comprising a NORI amino acid sequence of SEQ ID NO:64, a nucleic acid comprising a NOR1 nucleic acid sequence of SEQ ID NO:47, a polypeptide comprising a NUR77 amino acid sequence of SEQ ID NO:91, a nucleic acid comprising a NUR77 nucleic acid sequence of SEQ ID NO:76, or pharmaceutically acceptable salts thereof, polypeptides, peptides and/or agents, and/or gene therapy vectors, including both wild-type and/or antisense vectors, into host cells.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a preferred embodiment, of the invention, the pharmaceutical composition may be associated with a lipid. The pharmaceutical composition associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/pharmaceutical composition associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al, 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer and expression of an oligonucleotide in vitro and in vivo, then they are applicable for the present invention. Where a bacterial promoter is employed in the DNA construct, it also will be desirable to include within the liposome an appropriate bacterial polymerase.

Liposomes used according to the present invention can be made by differentmethods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000× g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

Gene Therapy Administration:

For gene therapy, a skilled artisan would be cognizant that the vector to be utilized must contain the gene of interest operatively limited to a promoter. For antisense gene therapy, the antisense sequence of the gene of interest would be operatively linked to a promoter. One skilled in the art recognizes that in certain instances other sequences such as a 3′ UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutic nucleic acid sequence must be administered to provide a pharmacologically effective dose of the gene product.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein the vector is complexed to another entity, such as a liposome or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene may also contain a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of the suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSVtk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The method of cell therapy may be employed by methods known in the art wherein a cultured cell containing a non-defective NURR1 nucleic acid sequence encoding a NURR1 protein is introduced.

In another embodiment, biologically active molecules, such as vectors for gene therapy, are incorporated in a large hydration domain between “pinched” regions of a lipid-poly-L-glutamic acid (PGA) complex, where the PGA and the cationic lipid didodecyl dimethylammonium bromide associate to form localized pinched regions, for delivery applications (Subramaniam, et al, 2000).

In an alternative embodiment, an amino acid sequence is engineered to accumulate as an aggregate in the endoplasmic reticulum, followed by administration of a composition to induce protein disaggregation, resulting in rapid and transient secretion (Rivera et al., 2000).

A peptide (11 amino acids) derived from HIV has been recently described that when fused to full length proteins and injected into mice allow a rapid dispersal to the nucleus of all cells of the body (Schwarze et al., 1999). Schwarze et al. made fusion proteins to Tat ranging in size from 15 to 120 kDa. They documented a rapid uptake of the fusion proteins to the nuclei of cells throughout the animal, and the functional activity of the proteins was retained.

In an embodiment of the present invention there are constructs containing the Tat or Tat-HA nucleic acid sequence operatively linked to a NURR subfamily nucleic acid sequence. The vectors are expressed in bacterial cultures and the fusion protein is purified. This purified Tat-HA-NURR subfamily protein or Tat-NURR subfamily protein is injected into animal to determine the efficiency of the Tat delivery system into the site of inflammation, the joints, or by means to deliver the fusion protein systemically. Analysis is carried out to determine the potential of the Tat-HA-NURR subfamily protein or Tat-NURR subfamily protein in reduction of inflammation or alleviation of any arthritis symptom. This is a viable therapeutic approach either in its own right or in association with other methods, treatments or genes.

DNA Delivery Using Viral Vectors:

The ability of certain viruses to infect cells via receptor-mediated endocytosis, to integrate into host cell genome and to express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles, endotoxins, and other pyrogens such that it will not cause any untoward reactions in the cell, animal or individual receiving the vector construct. A preferred means of purifying the vector involves the use of buoyant density gradients, such as cesium chloride gradient centrifugation.

a. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus vector” is meant to include those constructs containing adenovirus sequences sufficient to (a) support packaging of the construct and (b) to ultimately express a tissue or cell-specific construct that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and/or Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The El region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and/or propagation of the current adenovirus vectors, which are replication deficient, depend on a unique helper cell line, designated 293, which was transformed from embryonic kidney cells by Ad5 DNA fragments and constitutively expresses E1 proteins (E1A and/or E1B; Graham et al., 1977). Since the E3 region is dispensable from the adenovirus genome (Jones and Shenk, 1978), the current adenovirus vectors, with the help of 293 cells, carry foreign DNA in either the El, the D3 or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from mammalian cells such as human embryonic kidney cells, muscle cells, hematopoietic cells and other human embryonic mesenchymal or epithelial cells. Alternatively, the helper cells may be derived from the cells of other mammalian species that are permissive for adenovirus. Such cells include, e.g., Vero cells and/or other monkey embryonic mesenchymal and/or epithelial cells. As stated above, the preferred helper cell line is 293.

Recently, Racher et al. (1995) disclosed improved methods for propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and genetic information is known, and it has historically been used for most constructions employing adenovirus as a vector.

As stated above, the typical vector according to the present invention is replication defective and will not have an adenovirus El region. Thus, it will be most convenient to introduce the transforming construct at the position from which the E1-coding sequences have been removed. However, the position of insertion of the construct within the adenovirus sequences is not critical to the invention. The polynucleotide encoding the NURR subfamily member may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 109 to 1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and/or Horwitz, 1992; Graham and/or Prevec, 1992). Recently, animal studies suggested that recombinant adenovirus could be used for gene therapy (Stratford-Perricaudet and/or Perricaudet, 1991a; Stratford-Perricaudet et al., 1991b; Rich et aL, 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and/or Gerard, 1993) and stereotactic inoculation into the brain (Le Gal La Salle et al., 1993). Recombinant adenovirus and adeno-associated virus (see below) can both infect and transduce nondividing mammalian primary cells.

b. Adeno-associated Viral Vectors

Adeno-associated virus (AAV) is an attractive vector system for use in the cell transduction of the present invention as it has a high frequency of integration, and it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et aL, 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et aL (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and/or Muzyczka, 1984; Tratschin et al., 1985; McLaughlin et al., 1988) or genes involved in mammalian diseases (Flotte et al., 1992; Luo et al., 1994; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al, 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al, 1995).

c. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al, 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and/or Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, and env sequences integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, which normally infects only mouse cells, that modified an envelope protein so that the virus specifically bound to, and infected, mammalian cells bearing the erythropoietin (EPO) receptor. This was achieved by inserting a portion of the EPO sequence into an envelope protein to create a chimeric protein with a new binding specificity.

d. Other Viral Vectors

Other viral vectors may be employed as expression constructs in the present invention. Vectors derived from viruses such as vaccinia virus (Ridgeway, 1988; Baichwal and/or Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and/or Sugden, 1986; Coupar et aL, 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

e. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin (Roux et al., 1989). Using antibodies against major histocompatibility complex class I and class II antigens, they demonstrated the infection of a variety of mammalian cells that bore those surface antigens with an ecotropic virus in vitro (Roux et al., 1989).

Antibody Preparation

In certain aspects of the invention, one or more antibodies may be produced to the expressed NURR subfamily members and CRH receptors. These antibodies may be used in various diagnostic or therapeutic applications, described herein below.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof.

a. Polyclonal Antibodies

Polyclonal antibodies to the NURR subfamily members and to the CRH receptors generally are raised in animals by multiple subcutaneous (sc) or intraperitoneal (ip) injections of the NURR subfamily member or CRH receptor and an adjuvant. It may be useful to conjugate the NURR subfamily member or CRH receptor, or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹ N═C═NR, where R and R¹ are different alkyl groups.

Animals are immunized against the immunogenic conjugates or derivatives by combining 1 mg of 1 μg of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. 7 to 14 days later the animals are bled and the serum is assayed for anti-NURR anti-CRH receptor antibody titer. Animals are boosted until the titer plateaus. Preferably, the animal boosted with the conjugate of the same NURR subfamily member or of the same CRH receptor, but conjugated to a different protein or through a different cross-linking reagent. Conjugates also can be made in recombinant cell culture as protein fusions. Also, aggregating agents such as alum are used to enhance the immune response.

b. Monoclonal Antibodies

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, the anti-NURR or anti-CRH receptor monoclonal antibodies of the invention may be made using the hybridoma method first described by Kohler & Milstein, 1975, or may be made by recombinant DNA methods (Cabilly, et al., U.S. Pat. No. 4,816,567). In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC- 11 mouse tumors available from the Salk Institute Cell Distribution Center, San Diego, Calif. USA, and SP-2 cells available from the American Type Culture Collection, Rockville, Md. USA. Culture medium in which hybridoma cells are growing is assayed for production of monoclonal antibodies directed against a NURR subfamily member or a CRH receptor. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson & Pollard, 1980. After hybridoma cells are identified that produce antibodies of the desired specificity, affinity, and activity, the clones may be subcloned by limiting dilution procedures and grown by standard methods. Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (Morrison, et al., 1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-NURR or anti-CRH receptor monoclonal antibody herein. Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for a NURR subfamily member or a CRH receptor and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate. For diagnostic applications, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g. ¹²⁵I, ³²P, ¹⁴C, or ³H, or an enzyme, such as alkaline phosphatase, beta-galactosidase or horseradish peroxidase. Any method known in the art for separately conjugating the antibody to the detectable moiety may be employed, including those methods described by Hunter, et al., 1962; David et al., 1974; Pain, et al., 1981; and Nygren, 1982.

The antibodies of the present invention may be employed in any known assay method, such as competitive binding assays, direct and indirect sandwich assays, and immunoprecipitation assays. Zola, Monoclonal Antibodies: A Manual of Techniques, pp.147-158 (CRC Press, Inc., 1987). Competitive binding assays rely on the ability of a labeled standard (which may be a NURR subfamily member or a CRH receptor or an immunologically reactive portion thereof) to compete with the test sample analyte (NURR subfamily member or CRH receptor) for binding with a limited amount of antibody. The amount of NURR subfamily member or CRH receptor in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. David & Greene, U.S. Pat. No. 4,376,110. The second antibody may itself be labeled with a detectable moiety (direct sandwich assays) or may be measured using an antiimmunoglobulin antibody that is labeled with a detectable moiety (indirect sandwich assay). For example, one type of sandwich assay is an ELISA assay, in which case the detectable moiety is an enzyme.

(iii) Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers Jones et al., 1986); Riechmann et al, 1988; Verhoeyen et al., 1988, by substituting rodent CDRs or CDR sequences for the corresponding sequences of a human antibody. Accordingly, such “humanized” antibodies are chimeric antibodies (Cabilly, supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. Pat. No. 5,821,337 filed Oct. 13, 1998.

d. Human Antibodies

Human monoclonal antibodies can be made by the hybridoma method. Human myeloma and mouse-human heteromyeloma cell lines for the production of human monoclonal antibodies have been described, for example, by Kozbor, J. Immunol. (1984), and Brodeur, et al., Monoclonal Antibody Production Techniques and Applications, pp.51-63 (Marcel Dekker, Inc., New York, 1987). It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germline mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., 1993; Jakobovits et al, 1993. Alternatively, the phage display technology (McCafferty et al., 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., 1991 or Griffith et al., 1993.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This techniques allows the production of antibodies and antibody fragments with affinities in the nIM range. A strategy for making very large phage antibody repertoires (also known as “the mother-of-all libraries”) has been described by Waterhouse et al., 1993, and the high affinity human antibody can be directly isolated from the large phage library as previously reported. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published Apr. 1, 1993). Unlike traditional humanization of rodent antibodies by CDR grafting, this technique provides completely human antibodies, which have no framework or CDR residues of rodent origin.

e. Bispecific Antibodies

Bispecific antibodies are monoclonal, preferably human or humanized, antibodies that have binding specificities for at least two different antigens. In the present case, one of the binding specificities is for a NURR subfamily member or for a CRH receptor, the other one is for any other antigen, and preferably for another receptor or receptor subunit. For example, bispecific antibodies specifically binding a NURR subfamily member or a CRH receptor and neurotrophic factor, or two different NURR subfamily members or two different CRH receptors are within the scope of the present invention. Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, 1983). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published May 13, 1993), and in Traunecker et al., 1991.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment, of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in copending application Ser. No. 07/931,811 filed Aug. 17, 1992. For further details of generating bispecific antibodies see, for example, Suresh et al., 1986.

f. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT Application Publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Immunodetection Methods

In still further embodiments, the present invention concerns immunodetection methods for binding, purifying, removing, quantifying and otherwise generally detecting biological components such as a NURR subfamily member and CRH receptor subtype protein components. The NURR subfamily member and CRH receptor subtype antibodies prepared in accordance with the present invention may be employed to detect NURR subfamily member or CRH receptor proteins, polypeptides and peptides. As described throughout the present application, the use of NURR subfamily member and CRH receptor subtype specific antibodies is contemplated. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing a NURR subfamily member or a CRH receptor protein, polypeptide or peptide, and contacting the sample with a first anti-NURR or anti-CRH antibody in accordance with the present invention, as the case may be, under conditions effective to allow the formation of immunocomplexes.

These methods include methods for purifying NURR subfamily member and CRH receptor proteins, polypeptides and peptides as may be employed in purifying NURR subfamily member and CRH receptor proteins, polypeptides and peptides from patients' samples and for purifying recombinantly expressed NURR subfamily member or CRH receptor proteins, polypeptides and peptides. In these instances, the antibody removes the antigenic NURR subfamily member or CRH receptor subtype protein, polypeptide or peptide component from a sample. The antibody will preferably be linked to a solid support, such as in the form of a column matrix, and the sample suspected of containing the NURR subfamily member or CRH receptor protein antigenic component will be applied to the immobilized antibody. The unwanted components will be washed from the column, leaving the antigen immunocomplexed to the immobilized antibody, which NURR subfamily member or CRH receptor protein antigen is then collected by removing the NURR subfamily member or CRH receptor proteins or peptides from the column.

The immunobinding methods also include methods for detecting and quantifying the amount of a NURR subfamily member or a CRH receptor protein reactive component in a sample and the detection and quantification of any immune complexes formed during the binding process. Here, one would obtain a sample suspected of containing a NURR subfamily member or CRH receptor subtype protein or peptide, and contact the sample with an antibody against NURR subfamily member or CRH receptor subtype, and then detect and quantify the amount of immune complexes formed under the specific conditions.

In terms of antigen detection, the biological sample analyzed may be any sample that is suspected of containing a NURR subfamily member and/or CRH receptor subtype protein-specific antigen, such as an inflamed synovial tissue section or specimen, a homogenized inflamed synovial tissue extract, an inflamed synovial cell, separated and purified forms of any of the above NURR subfamily member or CRH receptor protein-containing compositions, or even any biological fluid that comes into contact with the inflamed synovial tissue, such as synovial fluid, although tissue samples or extracts are preferred. Inflammatory immune diseases that may be suspected of containing a NURR subfamily member or CRH receptor proteinspecific antigen include, but are not limited to, the collection of conditions classified as chronic inflammatory joint disease, ulcerative colitis, thyroiditis, arthritis, rheumatoid arthritis, psoriatic arthritis, and sarcoid arthritis.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any NURR subfamily member or CRH receptor protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The NURR subfamily member or CRH receptor antibodies employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immunoPCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various forms of inflammatory immune disease, such as rheumatoid arthritis. Here, a biological or clinical sample suspected of containing a wild-type or mutant NURR subfamily member or CRH receptor protein, polypeptide, peptide or mutant is used. However, these embodiments also have applications to non-clinical samples, such as in the titering of antigen or antibody samples, for example in the selection of hybridomas.

In the clinical diagnosis and/or monitoring of patients with various forms of inflammatory immune disease, such as rheumatoid arthritis, the detection of NURR subfamily member and CRH receptor protein, polypeptide, peptide or mutant, or an alteration in the levels of NURR subfamily member and CRH receptor, in comparison to the levels observed in a corresponding biological sample from a normal subject is, in a specific embodiment, indicative of a patient with inflammatory immune disease, such as rheumatoid arthritis. However, as is known to those of skill in the art, such a clinical diagnosis would not necessarily be made on the basis of this method in isolation. Those of skill in the art are very familiar with differentiating between significant differences in types and amounts of biomarkers, which represent a positive identification, or low level and background changes of biomarkers. Indeed, background expression levels are often used to form a “cut-off” above which increased detection will be scored as significant and positive.

a. Immunohistochemistry

The antibodies of the present invention may also be used in conjunction with both fresh-frozen and formalin-fixed, paraffin-embedded tissue blocks prepared for study by immunohistochemistry (IHC). The method of preparing tissue blocks from specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfiuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating or embedding the block in paraffin; and cutting up to 50 serial permanent sections.

Non-protein-expressing Sequences

In certain embodiments, the NURR subfamily nucleic acid sequence or CRH receptor may express messages that are not translated. DNA may be introduced into organisms for the purpose of expressing RNA transcripts that function to affect phenotype yet are not translated into protein. Two examples are antisense RNA and RNA with ribozyme activity. Both may serve possible functions in reducing or eliminating expression of native or introduced genes. However, as detailed below, DNA need not be expressed to effect the phenotype of an organism.

a. Antisense RNA

In certain aspects, a NURR subfamily nucleic acid sequence or CRH receptor may express an antisense message. Nucleic acids, particularly those from genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the cell's genome. The aforementioned genes will be referred to as antisense genes. An antisense gene may thus be introduced into a cell by transformation methods to produce a novel transgenic cell or organism with reduced expression of a selected protein of interest. For example, the protein may be an enzyme that catalyzes a reaction in the cell or organism. Reduction of the enzyme activity may reduce or eliminate products of the reaction which include any enzymatically synthesized compound in the cell or organism such as fatty acids, amino acids, carbohydrates, nucleic acids and the like.

The following examples are offered by way of example and are not intended to limit the scope of the invention in any manner.

### EXAMPLE 1

### Production of CRH mRNA in Inflamed Human Synovial Tissue

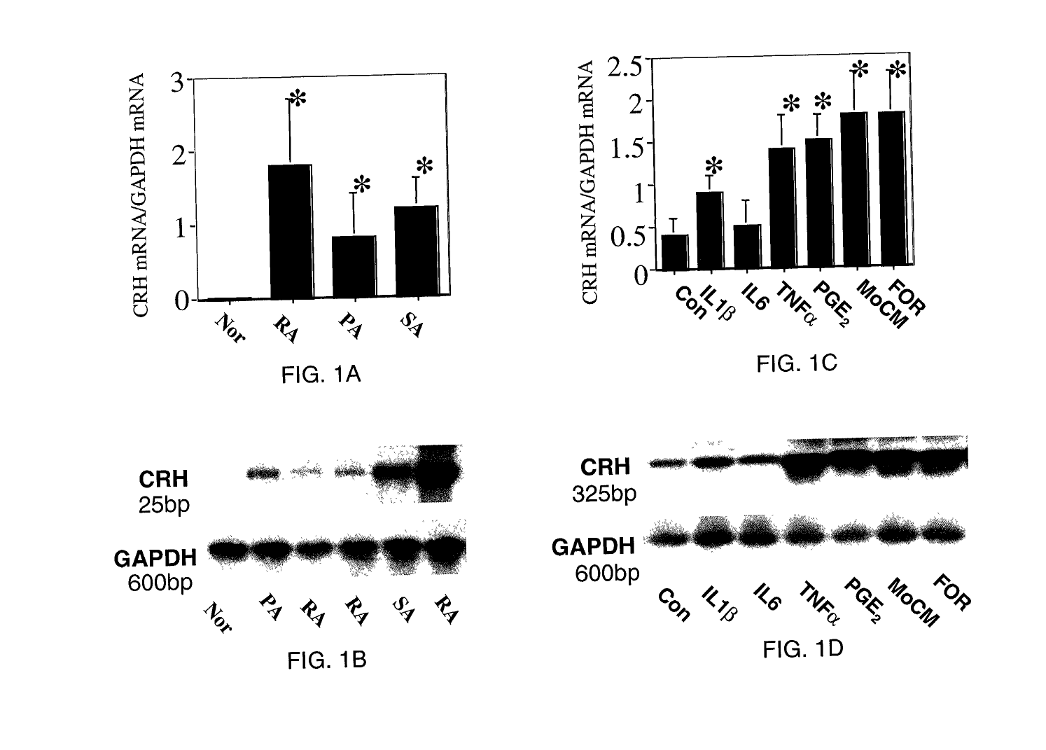

To examine wh ether CRH mRNA is exp ressed peripherally in inflamed human synovium RT-PCR was used to compare CRH mRNA levels in synovial tissue obtained from the joints of patients with RA (n=6, 1.80+/−0.87), PsA (n=6, 0.81+/−1.6), SA (n=2. 1.20+/−0.4) and normal synovium (n—2). All disease groups had significantly increased CRH mRNA levels (P<0.01) when compared to normal synovium (FIGS. 1A and B). Expression levels of the house keeping gene glutaraldehyde-3 -phosphate dehydrogenase (GAPDH) were similar in all patients.

Total RNA was analyzed by RT-PCR using primers to human CRH and GAPDH. (FIG. 1A) normal human synovium (Nor), patients diagnosed with psoriatic arthritis (PsA), rheumatoid arthritis (RA) and sarcoid arthritis (SA). (FIG. 1B) Values shown are the mean +/−SE. *indicates P<0.01 compared to normal synovium.

### EXAMPLE 2

### Expression of CRH in Primary Human Synoviocytes

To identify the origin of the cells that express CRH, the expression of CRH mRNA in isolated primary human synoviocyte cells was investigated. Synoviocytes are an important source and target cell for IL-1β, TNFα and PGE₂ action within inflamed synovial tissue (Bucala et al., 1991). Moreover, these pro-inflammatory agents are important mediators in the pathogenesis of human synovial hyperplasia and cartilage destruction (MacNaul et aL., 1990). RT-PCR analysis demonstrated that CRH MRNA was expressed constitutively in both RA and PsA primary synoviocyte cells. (FIG. 1C) Primary synoviocytes following stimulation by pro-inflammatory mediators are shown in FIG. 1D. Representative examples of PCR products generated using synoviocytes maintained under normal conditions (Con), or exposed to 10 ng/ml IL-1β, IL-6, TNF, 1 μM PGE₂, MoCM or 25 μM forskolin (FOR) for 6 h. Results were obtained from three separate experiments, using both RA and PsA cell lines. Values shown are the mean +/−SE. The symbol * indicates P<0.01 compared to control. PCR products generated were confirmed by Southern blot analysis using CDNA probes for human CRH and GAPDH.

### EXAMPLE 3

### Effect of Forskolin and Pro-inflammatory Agonists on CRH Expression

The regulation of hypothalamic CRH gene expression by activation of adenylate cyclase/protein kinase A signaling pathway has previously been established (Guardiola-diaz et al., 1994). The ability of forskolin (FOR), an adenylate cyclase activating agent, was tested for stimulatory effects on CRH gene expression in primary synoviocytes. Forskolin induced endogenous CRH mRNA levels as early as 2 h post stimulation with levels peaking at 6 h (FIGS. 1C and D). To determine regulatory signals of peripheral CRH expression the ability of pro-inflammatory agonists to regulate endogenous CRH MRNA was examined. Synoviocytes stimulated with monocyte conditioned media (MoCM), a complex combination of inflammatory mediators reported to accurately reflect in vivo cytokine concentrations (Uhlar et al., 1997), strongly stimulated (4.64+/−2.5-fold) CRH mRNA expression to levels comparable with forskolin treatment. Next, a comparison of the ability of individual pro-inflammatory mediators to induce CRH mRNA was performed. TNFα (3.6+/−2.0-fold), IL-1 (2.3+/−1.0-fold) and PGE₂ (3.85+/−1.5-fold) significantly upregulated CRH mRNA (P<0.01) however, IL-6 had little effect (1.5+/−1.2-fold) on CRH mRNA levels in these cells at any of the concentrations tested. Levels of GAPDH mRNA remained relatively constant under all conditions tested (FIG. 1D).

### EXAMPLE 4

### Inflammatory Mediators Enhance the Transcriptional Activity of the Human CRH Promoter in Primary Synoviocytes

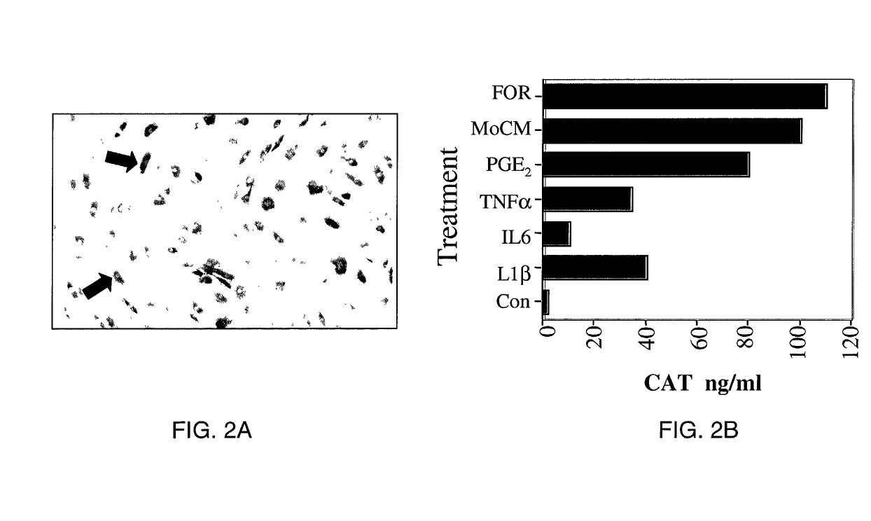

To determine whether inflammatory mediators are capable of regulating the expression of the human CRH promoter a reporter construct was generated by cloning the proximal promoter region (−666/+111) (SEQ ID NO: 143) of the human CRH gene into the promoterless pBL₃-chloramphenicol acetyl transferase (CAT) plasmid to create hCRH-CAT. Transcriptional regulation of the hCRH promoter was measured by transient transfection in primary human synoviocytes. In situ staining of the transfected cells for β-galactosidase activity revealed high transfection efficiencies of >95% (FIG. 2A). Three individual RA and PsA synoviocyte cell lines were transiently transfected with 3 μg -666 /+111 hCRH-CAT reporter plasmid to monitor CRH promoter activity, maintained under normal conditions (Con), or exposed to 10 ng/ml IL-1β, IL-6, TNFα1 μM PGE₂, MoCM or 25 μM FOR for 12 h. Consistent with the results of RT-PCR analysis TNFα (15.6+/−1.9-fold), IL-1 (17.1+/−3.1fold), PGE₂ (28.8+/−1.7-fold) and MoCM (40.8+/−2.1-fold) significantly (P<0.01) enhanced the transcriptional activity of the hCRH promoter. IL-6 treatment (10-100 ng/ml) of transfected cells did not significantly increase CAT production (3.2+/−1.9-fold) suggesting that IL-6 had little effect on CRH promoter activity in these cells. The responses of individual PsA and RA synoviocyte cell lines to treatment with proinflammatory agonists showed no significant differences. Representative CAT levels produced by transfected cells are illustrated in FIG. 2B.

The results shown in FIGS. 2A and 2B are representative of results from three RA and PsA individual cell lines. Each data bar represents two data points.

### EXAMPLE 5

### CRH Receptors are Expressed in Inflamed Synovium

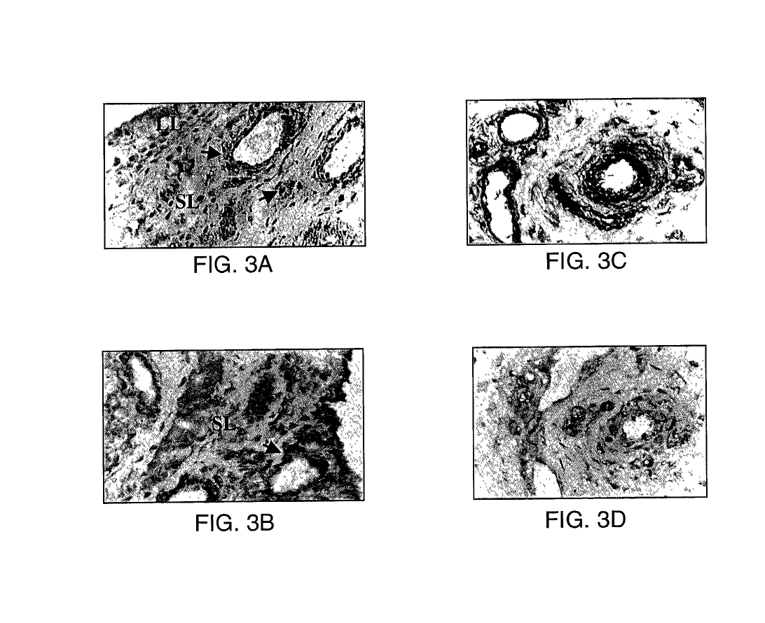

To further establish a peripheral biological role for CRH within inflamed human synovium the presence of CRH receptors was analyzed. Specific immunohistochemical staining for the CRH receptors was seen in all RA (n=6) and PsA (n=6) tissues examined. CRH receptors were present in the synovial vasculature, including endothelial cells and the smooth muscle layer of the blood vessels (FIG. 3). Receptor staining was consistently more intense in PsA (FIGS. 3B and C) compared to RA (FIG. 3A) synovial vasculature. Positive cells are indicated by brownish-black staining. LL indicates synovial lining layer, whereas SL indicates sublining synovial stroma. The original magnification is as follows: ×200 (A, B); ×450 (C, D). In contrast, although some unspecified mononuclear cells appeared to stain positive, the synovial lining layer, subsynovial synoviocytes and inflammatory infiltrates were predominately CRH receptor negative. Specificity of staining was verified by the absence of staining found on serial sections treated with CRH receptor antibody that had been pre-incubated with an excess of antigen (FIG. 3D).

Positive cells are indicated by brownish-black staining. The synovial lining layer, LL, and sublining synovial stroma, SL, are indicated. Original magnification: ×200 (A, B); ×450 (C, D). RA (A) and PsA (B, C and D) synovial tissue were stained with antibody directed against CRH receptors type 1 and 2 (C-20). PsA synovial tissue (D) (serial section to that in C) was stained with C-20 pre-absorbed with a C-20 specific blocking peptide.

### EXAMPLE 6

### NURR1 and NUR77 mRNA Expression in Synovial tissue

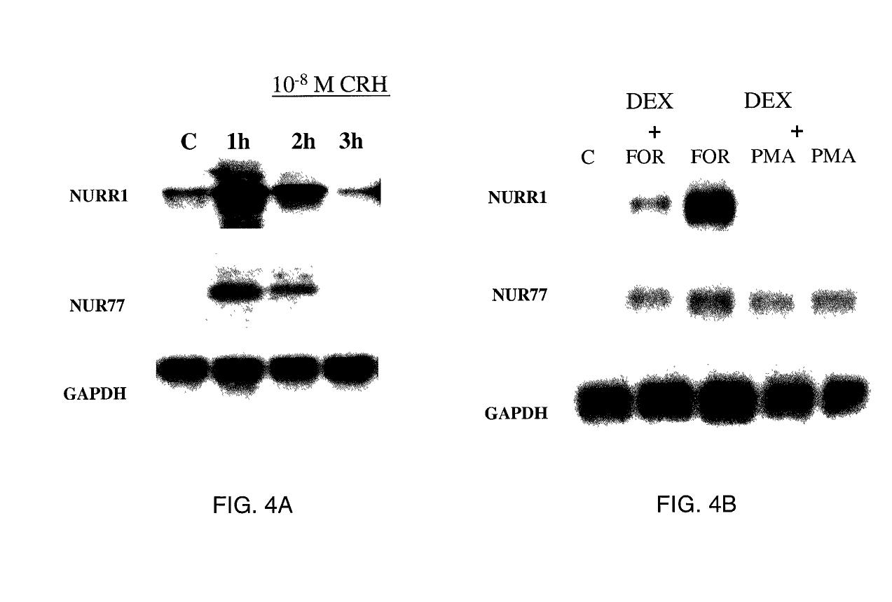

To determine whether the NURR subfamily contribute to CRH signaling in a pathological context, freshly excised RA (FIG. 4A) and PsA synovium explants were incubated with CRH (10⁻⁸ M) and examined for its ability to induce expression of NURR1 and NUR77 transcripts. Northern blot analysis confirmed that both NURR1 and NUR77 are rapidly induced by CRH within 1 h and decline to basal levels within 3 h (FIG. 4A). Furthermore, analysis of synovium explants indicates higher endogenous and CRH-inducible NURR1 mRNA compared to NUR77 transcript levels.

Regulation of gene expression by the protein kinase A and C pathways represent important signaling events in RA synoviocytes (Ben-Av et al., 1995; Crofford et al., 1994; Firestein and Manning, 1999). A rapid increase in NURR1 and NUR77 mRNA levels was observed following forskolin activation of protein kinase A pathways in these cells (FIG. 4B). To investigate the involvement of the protein kinase C signaling pathways in NURR regulation, synoviocytes were treated with phorbal myristic acetate (PMA). PMA modestly up-regulated NUR77 mRNA but had no stimulatory effects on NURR1 mRNA levels. Pre-treatment of synoviocytes with glucocorticoids (dexamethasone 10⁻⁸M) dramatically suppressed forskolin-induced NURR1 expression (FIG. 4B). In contrast, dexamethasone had little effect on forskolin- or PMA-induced NUR77 levels. Consistent with our immunohistochemical data, which suggests that synoviocytes do not express membrane CRH receptors, induction of NURR1 or NUR77 by CRH was not observed in primary synoviocyte cell lines. This observation indicates that the presence of CRH receptors are critical for the observed CRH-induced response on NURR1 expression.

In FIG. 4 total RNA was extracted and northern blot analysis was performed. Filters were hybridized with a cDNA for NURR1 or NUR77 and also with GAPDH to control for loading and transfer. Synovial explants were left untreated [C] or incubated with 10⁻⁸M CRH for the indicated times. (FIG. 4A) Confluent monolayers of primary synoviocytes were left untreated [C] or pretreated with 10⁻⁸M dexamethasone (DEX) for 2 h prior to the addition of 25μM forskolin (FOR) or 20 ng/ml of phorbal myristic acetate (PMA). (FIG. 4B)

### EXAMPLE 7

### Immunohistochemical Localization of NURR1 in Human Synovial Tissue

The demonstrated ability of CRH to induce synovial NURR expression suggests a regulatory role for the NURR transcription factors in human inflammatory arthritis. To test this hypothesis NURR1 expression was examined in both RA and PsA synovial tissue by immunohistochemical staining. Specific staining for NURR1 was seen in all RA (n=5) and PsA (n=5) tissues studied. Positive NURR1 staining was predominately nuclear and indicated by brownish-black staining.

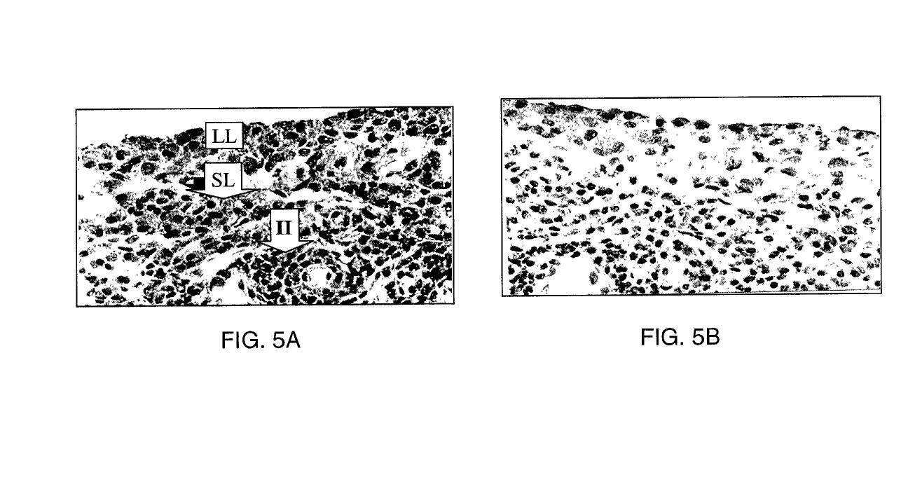

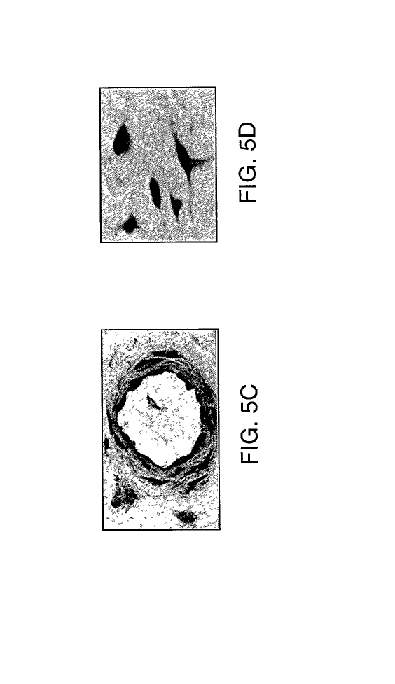

Staining revealed a predominately nuclear localization with some cytoplasmic NURR1 localization in cells of the synovial lining layer and the subsynovial region, which are areas primarily composed of synoviocyte and macrophage cells (FIG. 5A). Similarly, prominent mononuclear cell infiltrates in RA synovia were NURR1 positive. The synovial vasculature, including endothelial cells, was also a site for NURR1 expression with more intense staining observed in PsA synovial vessels (FIG. 5C). Immunohistochemical studies of NURR1 expression in cultured primary RA and PsA synoviocytes revealed a similar pattern of staining to that seen in vivo (FIG. 5D). The specificity of NURR1 staining was verified by the absence of staining seen when the NURR1 antibody was pre-incubated with an excess of antigen (FIG. 5B).

Synovial tissue sections represent RA synovial lining and sublining layers (FIGS. 5A and B) and PsA synovial vasculature (FIG. 5C) and cultured PsA synoviocytes (FIG. 5D). RA synovial tissue (B) (serial section to that in A) was stained with anti-NURR1 immune serum pre-absorbed with a NURR1 specific blocking peptide. The synovial lining layer, LL, the sublining synovial stroma, SL, and the inflammatory infiltrate, II, are indicated. Original magnification is as follows: ×200 (FIGS. 5A and B); ×1000 (FIGS. C and D).

### EXAMPLE 8

### Modulation of NURR1 Gene Expression by Inflammatory Agonists and Antagonists

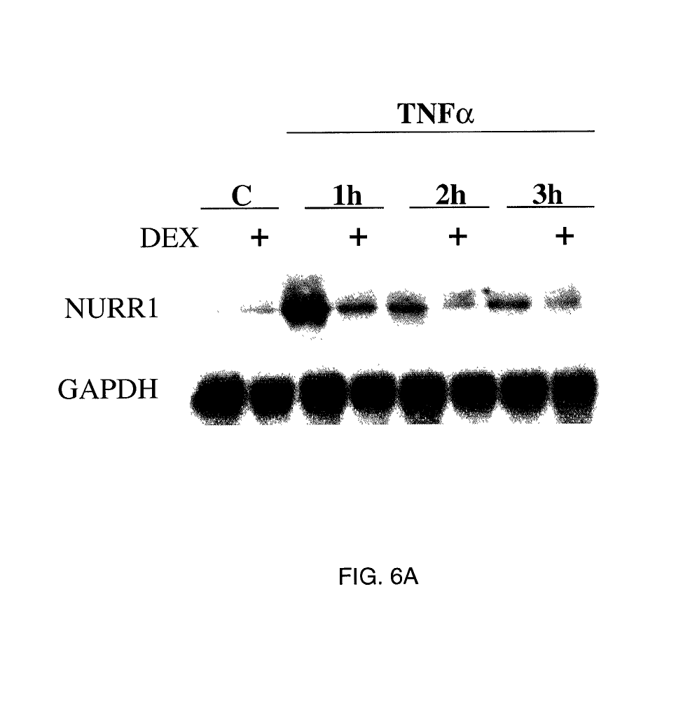

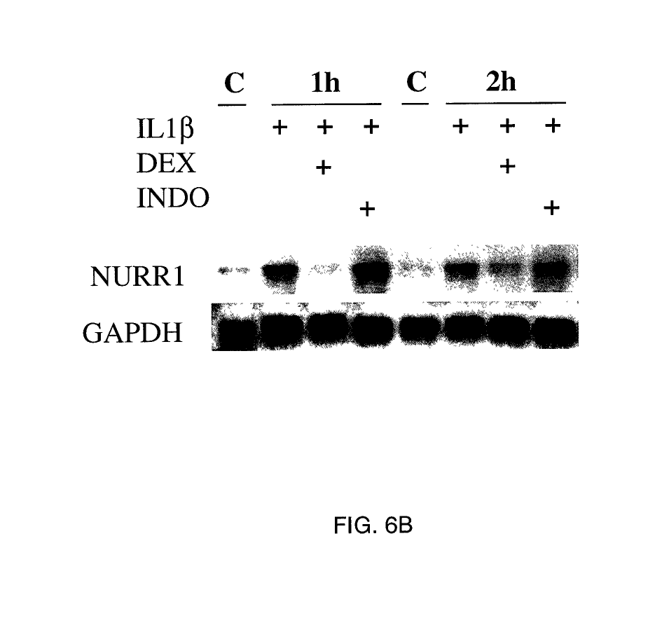

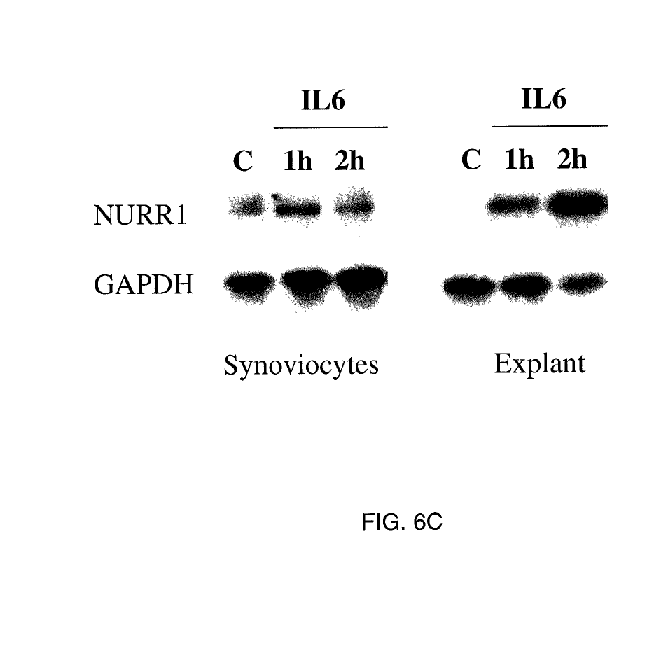

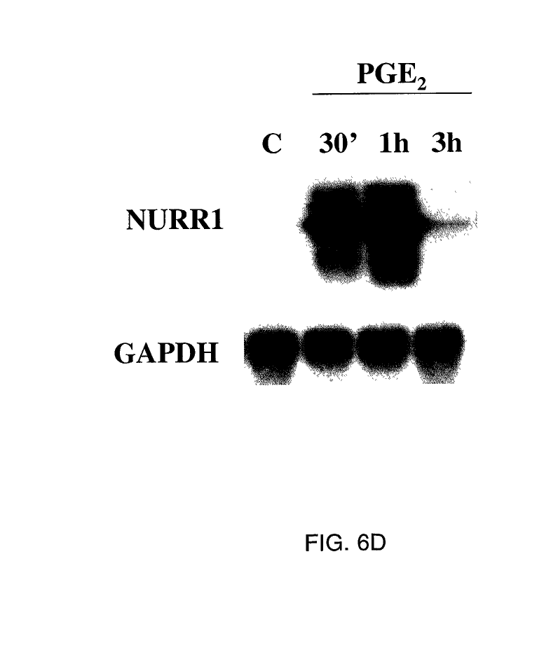

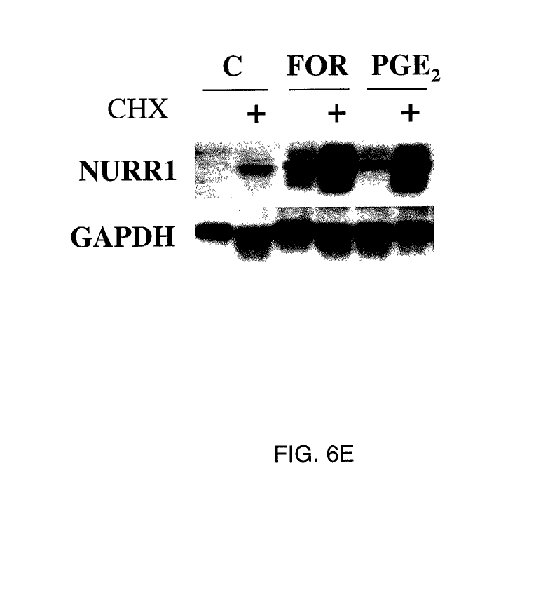

The extensive NURR1 staining observed in RA and PsA synovial tissue and in cells that do not express CRH receptors suggests that other mediators are also involved in the regulation of NURR1 expression. Therefore the pathological importance of the NURR transcription factors in inflammatory joint disease was further examined by determining the ability of pro-inflammatory agonists to regulate NURR transcript levels in primary synoviocytes and synovium explant tissue (FIG. 6). Treatment with TNFα, IL-1β, IL-6 and PGE₂ induced a rapid and marked increase in endogenous NURR1 mRNA levels (FIGS. 6A-D). Consistently, PGE₂ (FIG. 6D) had the most potent and sustained effect in stimulating NURR1 MRNA in these cells. In contrast to the stimulatory effects of IL-6 on NURR1 expression in isolated synovium explants, IL-6 treatment of synoviocytes did not significantly induce NURR1 MRNA (FIG. 6C).

Dexamethasone (10⁻⁸M) inhibited the TNFα, IL-1β (FIGS. 6A and B) and PGE₂-stimulated NURR1 MRNA in synoviocytes and CRH-induced NURR1 expression in synovium explants. In contrast, dexamethasone had no effect on basal NURR1 expression (FIG. 6A). IL-1β-induced NURR1 mRNA was not blocked by the cyclooxygenase inhibitor indomethacin, ruling out the involvement of autocrine PGE₂ action (FIG. 6B) (Ben-Av et al., 1994). Cycloheximide increased the endogenous NURR1 transcript levels and robustly enhanced the forskolin, PGE₂ (FIG. 6E), IL-1β and TNFαstimulation of NURR1 mRNA implying that de novo protein synthesis is not necessary for cytokine mediated induction of NURR1 mRNA in human synoviocytes. For each northern blot shown in FIG. 6, NUR77 mRNA levels were also analyzed. However, in contrast to the significant alterations in NURR1 mRNA levels, NUR77 transcript levels were only modestly regulated by each of the inflammatory agonists and/or antagonists studied. Notably, differential gene expression of synovial NURR1, NUR77 and NOR-1 was observed such that relative endogenous mRNA levels were NURR1 >>>NUR77 >>NOR-1.

For FIG. 6 each membrane was reprobed with a GAPDH cDNA to control for loading and transfer. NURR1 mRNA levels were measured by northern analysis using total RNA extracted from synoviocytes left untreated [C] or cultured for the time indicated with (A) TNFα (10 ng/ml) in the presence or absence of pre-treatment with 10⁻⁸M dexamethasone (DEX). (B) IL-1β(10 ng/ml) in the presence or absence of pretreatment with 2 μM indomethacin (INDO) or 10⁻⁸M DEX. (C) or synovial explants cultured with IL-6 (10 ng/ml). (D) 1 μM PGE₂. (E) 25 μM forskolin (FOR) or 1M PGE₂ for 1h in the absence or presence of 5 g/ml cycloheximide (CHX).

### EXAMPLE 9

### Increased Nuclear NURR1 Binding to the CRH NBRE Following Cytokine Treatment.

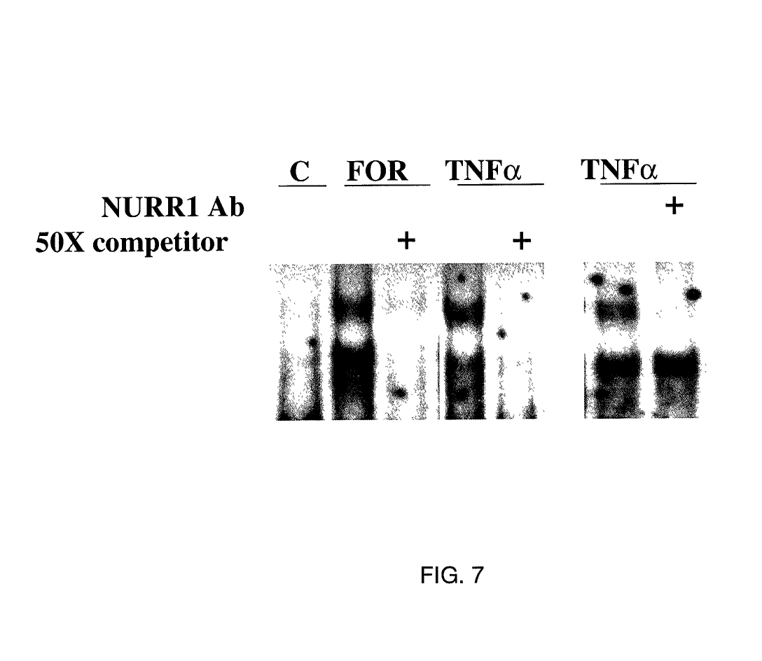

The stimulatory effect of pro-inflammatory cytokines on NURR1 gene expression demonstrates an important role of cytokines in the regulation of this transcription factor. To analyze the effects of cytokines on the ability of NURR1 to bind DNA, EMSA was performed to examine the binding properties of NURR1 to the consensus sequence (NBRE) in the human CRH promoter. Stimulation of synoviocytes with 25 μM forskolin, 10 ng/ml TNFα (FIG. 7), IL-1β or PGE₂ resulted in significant increased binding of two protein complexes with the CRH NBRE. Binding of the larger protein complex, and to a lesser extent the smaller complex, was inhibited by 50× molar excess of unlabeled homologous oligonucleotide. The larger protein complex was selectively blocked by NURR1 specific antiserum, previously shown to inhibit DNA binding by NURR1 (Murphy and Conneely, 1997), confirming that cytokine-induced increases in NURR1 mRNA correlate with specific binding of NURR1 protein to the CRH NBRE consensus sequence (FIG. 7).

Nuclear extracts from untreated, 25 μM forskolin (FOR) or lOng/ml TNFα treated synoviocytes (1 h) were compared for increased binding to the α³² P-labeled CRH NBRE. DNA-protein interactions were assayed in the presence of 50× molar excess of homologous oligonucleotide or NURR1 specific antiserum (NURR1 Ab).

### EXAMPLE 10

### The Role of Synovial CRH in Inflammatory Processes

These Examples have provided substantial evidence to support the conclusion that modulation of synovial CRH production is an important component of the inflammatory process associated with human inflammatory joint disease. The data demonstrate increased CRH mRNA in synovial tissue from patients with recent onset RA and other chronic inflammatory arthropathies including PsA and SA. In order to elucidate the regulatory mechanisms underlying peripheral CRH gene expression, the ability of pro-inflammatory mediators associated with joint inflammation and destruction to stimulate synovial CRH synthesis was tested. Endogenous expression of the CRH mRNA was confirmed by in vitro studies of cultured RA and PsA synoviocytes. These cells have been implicated in the synovial hyperplasia and the tumor-like invasive properties of synovium that leads to the cartilage destruction observed in human inflammatory arthritis (Bucala et al., 1991; MacNaul et al., 1990). It is also demonstrated herein that steady state CRH MRNA expression in primary synoviocytes was increased by IL1β, TNFα and PGE₂ and that the human CRH promoter responds to these same immunological stimuli in a manner similar to the response of endogenous CRH expression. It is possible that more than one cell type produces CRH mRNA in synovia. Immune cells of the rat spleen and thymus, mouse T lymphocytes and human peripheral blood lymphocytes have been reported to produce CRH mRNA (Webster et al., 1998), and thus infiltrating macrophages and lymphocytes in inflamed synovia also express CRH mRNA. However, the observation of a marked induction of CRH mRNA expression by synoviocytes supports the hypothesis that synoviocytes are a major source of CRH mRNA expression. Immunohistochemical localization of CRH receptors in synovium indicates that synovial CRH functions locally in a paracrine receptor-mediated response. Furthermore, the demonstration that CRH induces the nuclear transcription factors NURR1 and NUR77 in synovial explant tissue supports a significant role for the NURR subfamily in mediating the intracellular effects of synovial CRH. Taken altogether, these data provide evidence to support a local physiological role for peripheral CRH within the inflamed synovium.

### EXAMPLE 11

### The Significance of NURR1 in Mediating Multiple Inflammatory Signals

Several independent studies have implicated abnormal expression of nuclear immediate early genes, such as c-fos and c-jun, in the modulation of gene expression known to regulate cellular proliferation in RA synovium (Firestein et al., 1999). The Examples presented herein demonstrate, for the first time, NURR expression in inflamed synovial tissue and suggest these transcription factors contribute to the pathologic process of the disease. Endogenous NURR1 gene and protein expression in RA synovium, PsA synovium and proliferating synoviocytes was detected. Characteristic of immediate early response genes, the induction of NURR1 by IL-1β, TNFα, IL-6 and PGE₂ exhibited a rapid turnover rate independent of de novo protein synthesis. The ability of CRH to regulate NURR1 is analogous to NURR1 induction by established pro-inflammatory mediators and further underscores a peripheral role for CRH. Synovial-derived CRH induces NURR1 expression in CRH-receptor bearing cells. However, the abundant NURR1 expression in several synovial cell populations, that do not express CRH receptors, further supports a substantial role for the regulation of this transcription factor by the milieu of inflammatory mediators present in the inflamed joint. Consensus sequences for the binding of NF-κB and CREB in the human NURR1 proximal promoter are consistent with the induction of NURR1 expression in synovium by the immunological stimuli studied here. Investigation of NURR1 expression in primary synoviocytes indicates a role for NFκB in mediating both IL-1β- and TNFα-induced NURR1 transcription. These observations, together with previous demonstrations that CRH and PGE₂ signal by activation of cAMP/CREB dependent pathways (Labrie F., et al., 1982; Murphy and Conneely, 1997; Ben-Av et al., 1995), suggest that NURR1 induction represents a point of convergence of at least two distinct pro-inflammatory signaling pathways and an important common role for NURR1 in mediating multiple inflammatory signals.

### EXAMPLE 12

### Pathological Importance of the NURR Transcription Factors in Inflammatory Joint Disease

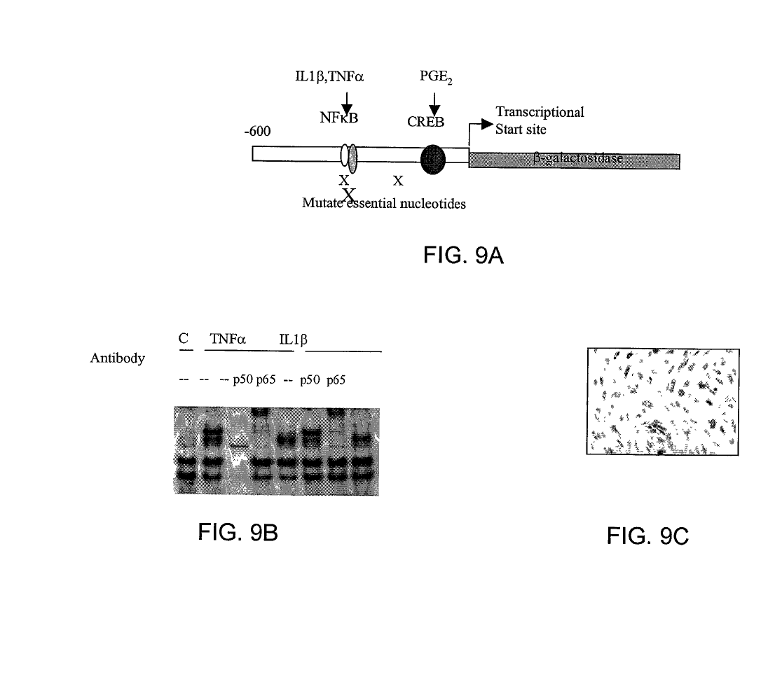

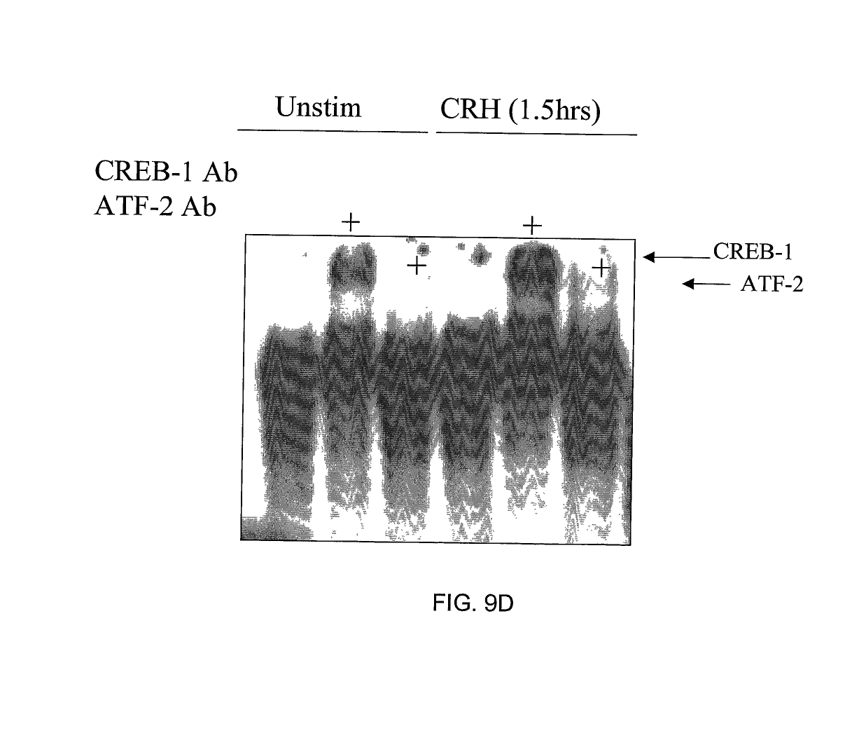

The pathological importance of the NURR transcription factors in inflammatory joint disease was examined by determining the ability of proinflammatory agonists, produced locally in RA, to regulate NURR transcript levels in primary human synoviocytes and synovium explant tissue. As demonstrated herein, treatment with TNFα, IL-1β, IL-6 and PGE₂ induced a rapid and marked increase in endogenous NURR1 mRNA levels. Consistently, PGE₂ had the most potent and sustained effect in stimulating NURR1 mRNA in these cells. It is well established that IL-1β and TNFα mediate transcription coupling through NF-κB, while PGE₂ signals by activation of CREB dependent pathways (5). Potential consensus sequences have been identified for both the binding of NF-κB and CREB in the human NURR1 proximal promoter (FIG. 9A). Electrophoretic mobility shift analysis (EMSA) was carried out to confirm that NFκB proteins are present in the cytokineinducible complexes of the NURR1 promoter (FIGS. 9B and 9D). To confirm the identity of the protein complexes binding to the NURR1 NFκB consensus site, supershift assays were performed using antibodies (p50 and p65) specific to NFκB members (FIG. 9B). Similarly, to confirm the identity of the protein complexes binding to the NURR1 consensus site, supershift assays were performed using antibodies (CREB-1 and ATF-2) specific to CREB members (FIG. 9D). These data suggest a novel role for NFκB and CREB in mediating pro-inflammatory induced NURR1 transcription. Primary RA synoviocytes were transfected with CMV-βgalactosidase reporter and stained for β-galactosidase activity to determine transfection efficiency. (FIG. 9C) Nuclear extracts from cultured synoviocytes (FIG. 9), untreated (c), treated with 10 ng/ml TNFα or IL1β for 1 hour, or treated with CRH for 1.5 hours were prepared and used in EMSA with oligonucleotide corresponding to the NFκB binding sequence (FIG. 9B) or oligonucleotide corresponding to the CREB binding sequence (FIG. 9D) of the NURR1 promoter. DNA-protein interactions were assayed in the presence of specific antibodies to the NFκB subunits p50 and p65 (FIG. 9B) or to CREB-1 and ATF-2 (FIG. 9D).

### EXAMPLE 13

### NURR Transcriptional Activity Contributes to Synovial Hyperplasia

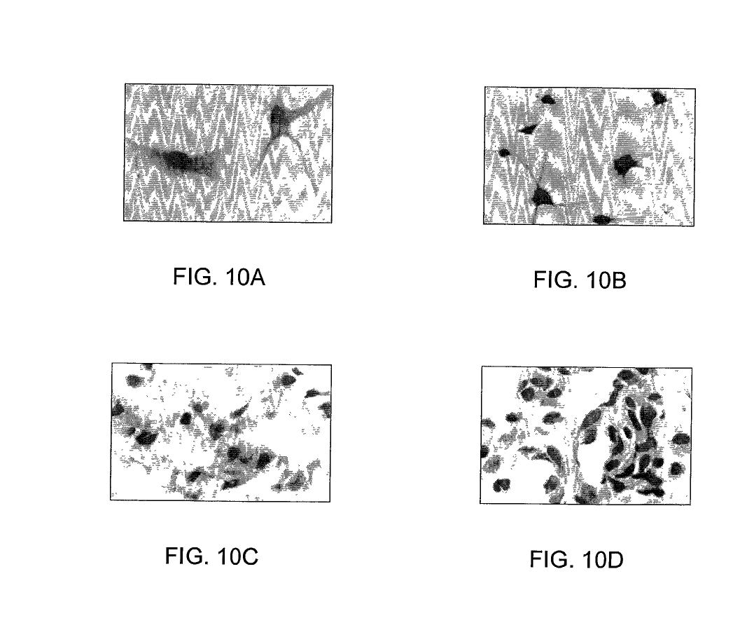

Evidence suggests that partial transformation of RA synoviocytes increases the invasive potential of RA synovium compared with osteoarthritis (OA). This unique rheumatoid phenotype is due to cytokine-regulated changes in transcription factor function and downstream target gene expression. Previous studies have reported that cytokine-stimulated release of resorptive agents such as MMPs and PGE₂ by synoviocytes occurs in association with a change from fibroblast-like to stellate morphology. Our analysis indicates that cytokine induction of NURR1 also occurs in parallel with a transformation of synoviocytes from a fibroblast-like to stellate shape (FIGS. 10A and B). Immunohistochemical staining was effected in primary PsA synoviocytes that were left untreated (A) or stimulated with TNFα (B) to illustrate the transformation from a fibroblast-like shape (A) to stellate shape (B) and the associated NURR1 nuclear localization. FIGS. 10C and D illustrate similar staining of normal synovium (C) and PsA synovial tissue (D) with anti-NURR1 immune serum which also shows a predominant nuclear localization of NURR1. Taken together, the results highlight the important in vivo transcriptional regulatory role of NURR1. Dexamethasone, an agent that inhibits cytokine-stimulated release of IL6, MMPs and PGE₂ by synoviocytes, also inhibits NURR1 induction and the cytokine-stimulated morphological transformation. These findings suggest that increased NURR1 gene expression is a component of a programmed change in gene expression that occurs when synoviocytes are activated to secrete inducible proinflammatory mediators of bone resorption.

### EXAMPLE 14

### CRH Receptors

An important process in chronic synovitis is the ability of locally produced cytokines to activate changes in the synovial vascular endothelium resulting in increased angiogenesis, vasodilation and plasma extravasation. CRH, in receptormediated response, has been implicated in enhancing local angiogenesis (Arbiser et al., 1999) and acts as a potent vasodilator by increasing vascular permeability at sites of inflammation (Theoharides et al., 1998). The findings presented herein, that CRH receptors are present in both the synovial vascular endothelium and the vascular smooth muscle, suggests that CRH participates in vascular processes associated with inflamed synovium. Two distinct subtypes of CRH receptors, CRH-R1 and CRH-R2, have been isolated and characterized (Aguilera G. et al., 1987; Perrin et al., 1995). The observations herein demonstrate a predominance of CRH RI expression in synovium, indicating that CRH R1 is more important than CRH R2 in mediating the effects of synovial CRH. The co-localization of CRH receptors and NURR1 in the endothelial vasculature further illustrates the role for this transcription factor in mediating synovial CRH responses. CRH receptor and NURR1 expression were consistently higher in the PsA synovial vasculature, supporting the theory that vascular changes are more pronounced in PsA compared to RA (Veale et al. 1993).

### EXAMPLE 15

### The Role of CRH R1α in the Mediation of Inflammatory Arthritis

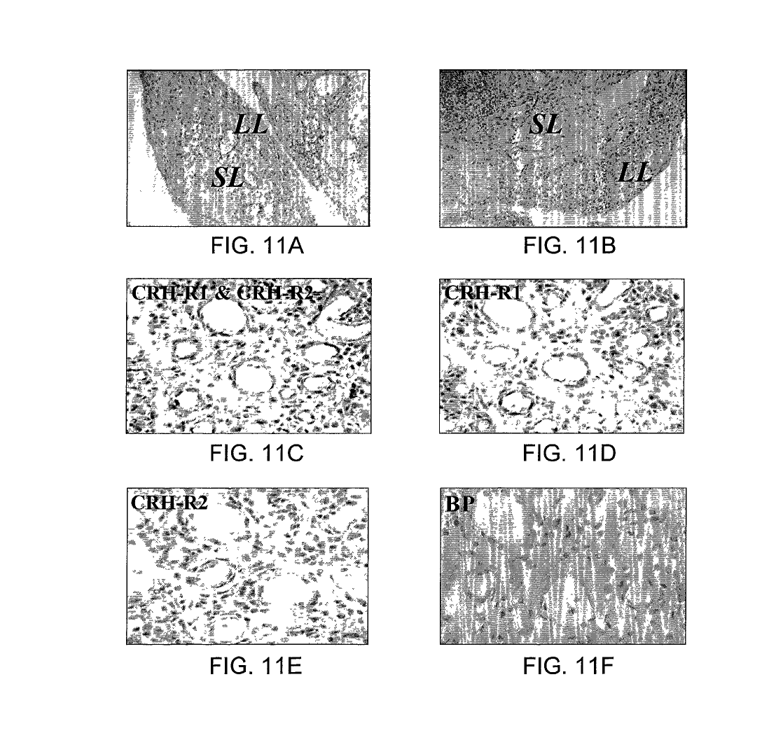

CRH receptors comprise seven putative membrane-spanning domains and belong to the calcitonin/vasoactive intestinal growth hormone releasing hormone subfamily of G protein-coupled receptors (Aguilera et al., 1987). Two distinct subtypes of CRH receptors, CRH-R1 and CRH-R2, have been isolated and characterized in the human central nervous system and are 68% homologous. Each subtype exhibits two alternatively spliced variants, displaying pharmacologically and functionally distinct isoforms (α and β) and exhibit distinct central and peripheral tissue specific expression patterns. Data reported herein indicate that CRH recepto-rmediated effects contribute to the pathogenesis of inflammatory diseases. Nevertheless, little information is available concerning CRH receptor status in pathologic inflammatory lesions. To determine whether CRH-receptor mediated responses contribute to the pathophysiology of inflammation in early human arthritis, the expression and localization of CRH receptors in RA (n=5) and PsA (n=9) synovial tissue was examined. (FIG. 11)

Immunohistochemical analysis was performed using immune serum that reacts with both CRH receptor subtypes CRH-R1 and CRH-R2 (FIG. 11). Considerably enhanced expression of CRH receptors was observed in the synovial vasculature including endothelial cells, and moderate expression in perivascular regions from all inflammatory arthritis patients (FIGS. 11A and 11B). The synovial lining layer, subsynovial synoviocytes and inflammatory infiltrates were consistently CRH receptor negative. CRH receptor expression was consistently more intense in PsA compared to RA synovial vasculature. Normal synovium did not express either receptor subtype.

### EXAMPLE 16

### Immunohistochemistry of CRH Receptor Subtype Expression in Human Synovial Tissue

Synovial biopsy samples from early inflammatory arthritis patient cohort (RA, n=5 and PsA, n=9) were further evaluated to determine if the receptor expression observed was due to the CRH-R1 or -R2 receptor subtype. Similar to the overall CRH-R1 and -R2 expression patterns (FIGS. 1A and B), distinct CRH-R1 staining was observed on vascular endothelium and other discrete perivascular cells (FIG. 11D). In contrast, specific CRH-R2 immune serum failed to detect CRH-R2 expression in the synovial tissue of the same patient cohort positive for CRH-R1 (FIG. 11E). The specificity of CRH receptor staining was ensured by a marked absence of staining found on serial sections treated with CRH receptor antibody that had been pre-incubated with an excess of specific antigen (FIG. 11F). Immunostaining with isotype matched non-immune goat IgG was negative in all RA and PsA synovial tissue examined. Histologically normal synovial tissue did not express either CRH-R1 or -R2 receptor subtypes (n 2).

### EXAMPLE 17

Dual Immunolocalization of Mast Cell Tryptase and CRH-R1

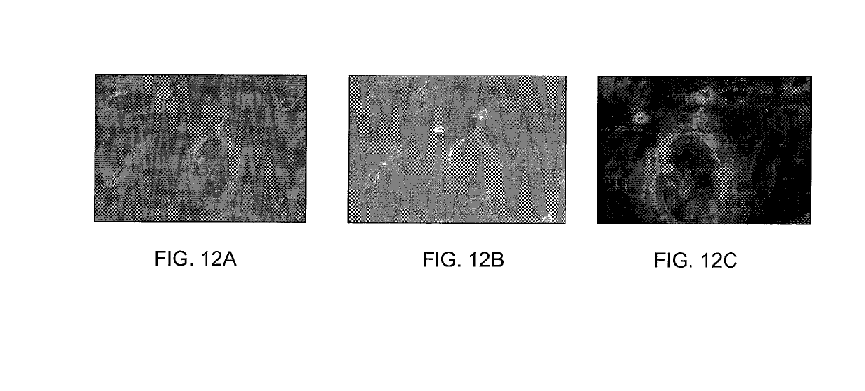

To identify the perivascular cells expressing immunoreactive CRH-Rl, synovial biopsy sections were immunostained using a double antibody staining method with anti-mast cell tryptase antibody and anti-CRH-R1 antibody (FIG. 12). Staining with anti-CRH-R1 antibody showed intense red fluorescence detected on vascular endothelium and discrete perivascular cells (FIG. 12A). Staining the same section with anti-mast cell tryptase antibody showed distinct green fluorescence (FIG. 12B). FIG. 12C illustrates dual immunolocalization of MC tryptase with CRH-R1 receptors on perivascular cells in inflamed PsA synovial tissue, thereby establishing that CRH-R1 is localized to perivascular mast cells in inflamed synovial tissues. A similar pattern of expression was detected in RA synovium.

### EXAMPLE 18

### Characterization of CRH Receptor Subtype mRNA Expression in Synovial Tissue

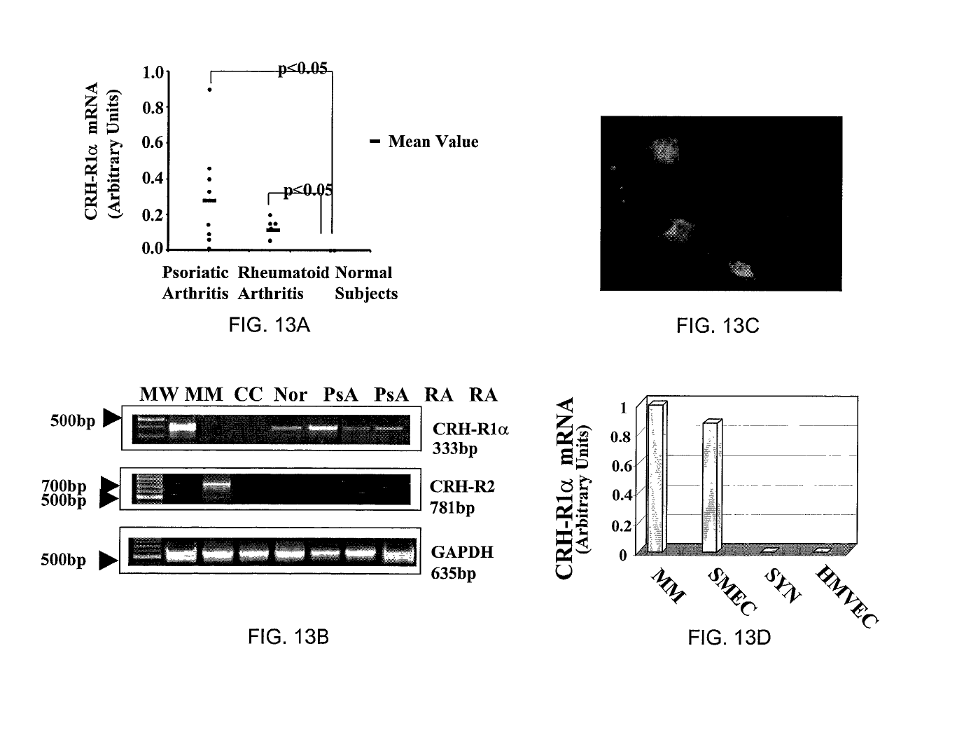

RT-PCR analysis was employed to characterize CRH receptor subtype MRNA expression in a cohort of human inflammatory arthropathies (RA, n=5 and PsA, n=8) (FIG. 13). Expression of both CRH-R1 and CRH-R2 receptor subtypes and their respective isoforms were examined. Amplification of cDNA with CRH-R1 primers predicted a fragment of 333 base pairs in length representing CRH-R1α and 420 base pairs in length representing the CRH-R11 isoform. CRH-R1α was detected in inflamed synovial tissue following amplification of reverse-transcribed mRNA in all inflammatory arthritis patients. In contrast, these primers did not detect the CRH-R1β isoform in synovial tissue from the same patient cohort. All RA and PsA synovial tissue samples had increased CRH-R1α MRNA levels when compared to normal synovium (p<0.05) (FIG. 13A). In accordance with immunohistochemical analysis, CRH-R1 MRNA levels were generally higher and more widely distributed in patients with PsA compared to patients with RA. FIG. 13B shows representative RT-PCR products generated for CRH-R1α receptor MRNA, using synovial tissue from two RA and two PsA patients. Histologically normal synovium did not express either CRH-R1 receptor isoforms.

CRH-R2 subtype expression was investigated in the same cohort of patients with arthritis (RA, n=5 and PsA, n=8). Specific CRH-R2 primers failed to amplify CRH-R2 mRNA in both inflamed and normal synovial tissue. To ensure the efficacy of the CRH-R2 primer pair, human cerebral cortex cDNA served as a positive control. Following reverse-transcription of human cerebral cortex MRNA, the predicted cDNA product of 781 bp was amplified using specific CRH-R2 primers (FIG. 13B). Expression levels of the housekeeping gene glutaraldehyde-3-phosphate dehydrogenase (GAPDH) were similar in all patients (FIG. 13B).

CRH receptor subtype expression in isolated synovial cell populations revealed a similar pattern of staining to that seen in vivo. Abundant CRH-R1 protein was localized to primary synovial endothelial cells (FIG. 13C). In contrast, neither CRH receptor subtypes were expressed on primary cultures of RA or PsA synoviocytes. Consistent with the immunolocalization data, high levels of CRH-R1α mRNA were present in primary synovial membrane endothelial cells (SMECs) but undetectable in RA and PsA primary synoviocytes (n=3) or in normal human microvascular endothelial cells (FIG. 13D). Expression of CRH-R2 mRNA was undetectable in all cell types investigated.

The observation of a marked induction of CRH-R1α (mRNA and protein) expression by synovial membrane endothelial cells supports the hypothesis that the vascular endothelium is a major target of CRH action and further suggests that CRH participates in vascular changes associated with synovial inflammation in RA. A rapid and efficient protocol for the isolation of microvascular endothelial cells (SMECs) from human RA synovial tissue is described in Example 25. Primary SMECs are microvascular endothelial cells making them ideal for studies on angiogenesis, a microvascular process. Initial studies of CRH receptor subtype expression in primary SMECs reveals a similar pattern of staining to that seen in vivo.

### EXAMPLE 19

### The Role of NURR1 as a Transcription Factor

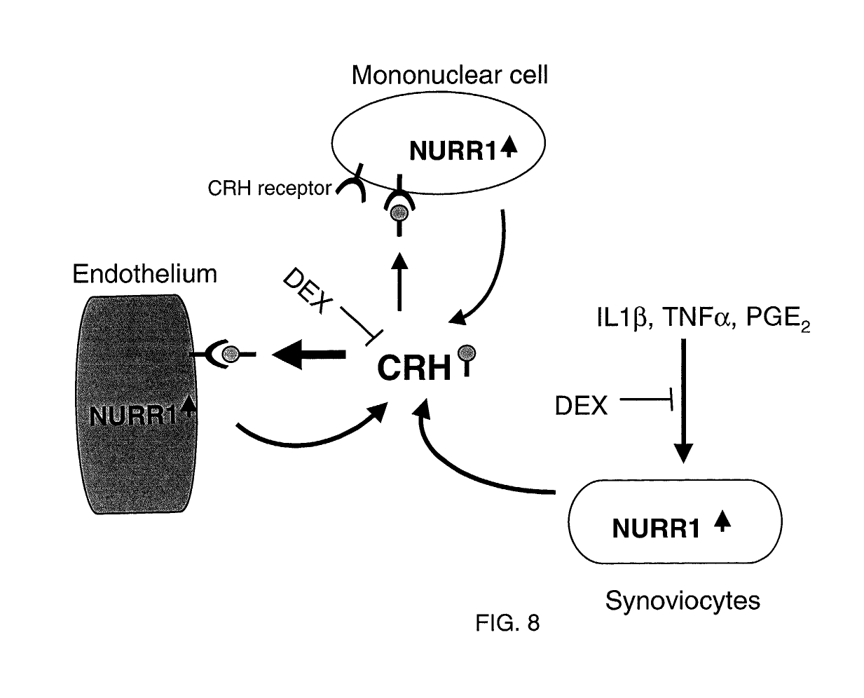

The predominant nuclear localization of synovial NURR1 in vivo highlights the important transcriptional regulatory role of NURR1. Synovial NURR1, directly regulated by locally produced cytokines, is a general mediator of an autocrine regulatory inflammatory cascade which serves to amplify the inflammatory response by increasing synovial CRH expression (FIG. 8). Immunohistochemical localization of NURR1 in the synovial lining layer, subsynovial synoviocytes and infiltrating mononuclear cells confirms that NURR1 is produced at the same synovial sites previously shown to express immunoreactive CRH (Crofford et al., 1993). The proximal promoter of the CRH gene contains an NBRE consensus sequence and the in vitro results confirm that cytokine-stimulated NURR1 mRNA levels correlate with increased nuclear binding of NURR1 protein to this consensus sequence. Evidence that POMC and POMC cleavage products (ACTH and β-endorphin) are expressed within inflamed synovia in the same cells that produce NURR1 and NUR77 suggests that the NURR subfamily are likely to regulate POMC expression in synovia (Fearon et al,. 1998). These observations are consistent with the earlier findings that CRH induction of pituitary POMC is mediated through the transcriptional activity of NURR1 and NUR77 (Murphy and Conneely, 1997). Furthermore, the cytokine responsive genes collagenase (Angel P., Baumann I., Stein B. Delius et al., 1987) and serum amyloid A (Uhlar et al,. 1997), which are expressed in human synoviocytes and have been implicated in RA inflammatory mechanisms and joint destruction, contain a NBRE consensus site in their proximal promoter regions. These findings lend further support to the theory that NURR1 and NUR77 are important transcriptional mediators of both CRH and pro-inflammatory cytokine responses in the pathogenesis of inflammatory joint disease. However, the differential gene expression of synovial NURR1 and NUR77 reveals the need for further studies to dissect the transcriptional roles of the individual NURR proteins (Murphy et al., 1995).

The inflammation of RA is extremely sensitive to glucocorticoids, which are used effectively in the treatment of rheumatoid synovitis. Thus, the findings reported herein that dexamethasone inhibits cytokine-induced NURR1 expression, together with the previously reported demonstration that dexamethasone significantly reduced immunoreactive levels of increased peripheral CRH in animal models of induced arthritis (Crofford et al., 1992), further highlights the pathological importance of these proteins in synovial tissue. The ability of CRH to signal locally suggests that synovial CRH participates in paracrine regulatory loops to facilitate communication between synoviocytes, endothelial and mononuclear cells. Immunoneutralization of CRH at peripheral inflammatory sites in vivo significantly inhibits inflammation and is analogous to the anti-inflammatory effects observed with TNFα antibodies (Karalis et al., 1991). Clinical trials and animal studies evaluating the potential of TNF blocking strategies and other anti-cytokine agents in the treatment of inflammatory arthritis disease activity suggest that multiple agents within the cytokine cascade is targeted to prevent disease progression and joint destruction. The data presented herein demonstrate that CRH induction occurs downstream to TNFα and IL-1β and therefore is an additional target for anti-cytokine therapy in human inflammatory arthritis. The in vivo evaluation of a CRH R1 antagonist, antalarmin, indicates that it provides a clinically effective CRH antagonist at sites of inflammation and have therapeutic promise in controlling local inflammation in diseases such as inflammatory arthritis where the pathophysiology involves CRH hyper-secretion (Webster et al., 1998).

### EXAMPLE 20

### Summary of the Roles of CRH and NURR1 in Inflammatory Arthritis

In summary, the data provided in this study indicate that modulation of locally produced CRH is an important component of the cytokine network in human inflammatory arthritis. Important mediators of inflammatory arthritis enhance the transcriptional activity of the CRH promoter and stimulate increased production of CRH gene expression in synovial cells that directly invade cartilage and bone. The presence of CRH receptors in inflamed synovium indicates peripheral CRH functions locally. The nuclear receptors NURR1 and NUR77 contribute to peripheral CRH signaling in human synovial tissue. The potential pathophysiological role for peripheral CRH and CRH-mediated pathways at sites of inflammation supports further investigation of antagonistic analogs of CRH as therapeutic agents in human inflammatory arthritis.

Furthermore, modulation of the NURR subfamily is an important mechanism regulating pathways associated with inflammatory joint disease, and observations suggest a central role for NURR1 in mediating multiple inflammatory responses. Signal transduction pathways involved in inflammation and cell transformation and their relationship to rheumatic diseases is a relatively unexplored research area. Aberrant function of transcription factor activity helps convert a normal phlogistic or immune response to a chronic state. In RA, inflammation propagates more inflammation, and therapeutic approaches to interrupt the perpetuation could provide an opportunity to reestablish homeostasis. The identification of molecular signaling pathways regulated by the NURR subfamily provides new approaches for intervention using the transcription factors as molecular targets of drug therapy. The vast potential of NURR1 family members as drug targets in medicine has already been proven in the case of the estrogen and androgen receptors. Clinical trials and animal studies evaluating the potential of TNFα blocking strategies and other anti-cytokine agents in the treatment of inflammatory arthritis disease activity suggest that multiple agents within the cytokine cascade need to be targeted to prevent disease progression and joint destruction. The data demonstrated herein that NURR1 induction occurs downstream to TNFα and IL-1β and therefore is a more effective target for anticytokine therapy in human inflammatory arthritis.

### EXAMPLE 21

### NFκB and CREB Binding to the NURR1 Promoter

Similar EMSAs and supershift assays using the NURR1 CREB consenus site performed with nuclear extracts from untreated or PGE₂-treated primary synoviocytes are performed. In addition in vivo levels of NFκB and CREB binding to the NURR1 promoter in nuclear extracts from both RA and PsA synovium compared to osteoarthritis (OA) synovium is measured. To determine whether NFκB and CREB binding to the NURR1 promoter is associated with disease activity, the relationship between relative density of EMSA NFκB and CREB activity in synovial tissue with clinical measurements of disease activity is measured.

To analyze the relative contribution of the consensus sites to cytokinemediated NURR1 transcription coupling and to further elucidate the mechanisms involved in these transduction pathways, a target gene construct containing 600 bp of the proximal human NURR1 promoter region fused to a β-galactosidase reporter gene was generated (FIG. 9A). The NURR1 promoter fragment used contains all of the sequences known to be necessary for correct expression of a β-galactosidase reporter gene when expressed in transgenic animals. Individual mutational analysis of the NFκB and CREB consensus sites within the NURR1 promoter permits evaluation of the role these consensus sites play in NURR1 expression. The wild type and mutated promoter constructs are individually transfected into primary RA and PsA synoviocytes and stimulated with the appropriate cytokine to monitor NURR1 transcriptional activity. Data described in Example 11 indicates that antiinflamrnatory agents such as dexamethasone can dramatically suppress cytokineinduced NURR1 mRNA levels. Thus, the transcriptional regulatory studies are extended to test the ability of dexamethasone to suppress cytokine induction of NURR1 transcription. This transfection assay system together with EMSAs is essential in identifying the mechanism of action of such drugs used in the treatment of RA.

### EXAMPLE 22

### NURR1 Mediates Synoviocyte Proliferation

NURR1 mediation of synoviocyte proliferation is evaluated by observing the consequences of NURR1 transient overexpression in primary RA and PsA synoviocyte cells and establishing stable NURR1-expressing transfectants using the human synoviocyte K41 cell line. The human NURR1 CDNA encoding the full length NURR1 protein is subcloned into the pUV6/V5-His A vector (Invitrogen) designed for overproduction of recombinant proteins in mammalian cells. The pUB6/V5-His vectors contain the blasticidin resistance gene to allow for selection of stable cell lines. Synoviocyte proliferation is measured using the standard CellTiter Assay (Promega).

Experimental approaches address the gene regulatory role of NURR1 and identify target genes that are regulated by this transcription factor in synovial tissue. The cytokine responsive genes MMP-1 and serum amyloid A (SAA), which have been implicated in RA inflammatory and destructive mechanisms, contain a putative NURR1 consensus binding site (NBRE) in their proximal promoter regions. These findings lend further support to the theory that NURR1 is an important transcriptional mediator of pro-inflammatory cytokine responses involved in the pathogenesis of inflammatory joint disease. Similar experimental approaches using EMSA and cell based transactivation assays are examined to determine NURR1 regulation of the synovial expression of MMP-1 and SAA by interacting with specific NBRE sequences.

Evidence suggests that NURR1 acts in an autocrine manner to stimulate the production of proinflammatory mediators. To further identify the pathophysiological pathways regulated by NURR1 transcriptional activity, the cell-based NURR1 transfection assay system is used to determine the involvement of NURR1 in the induction of inflammatory mediators (IL1α and TNFβ) and effector mediators of bone destruction (PGE₂, IL6 and MMPs). The levels of these mediators produced by NURR1-transfected primary synoviocytes is measured using individual PsA and RA synoviocyte cell lines. Such data provides further insights into these similar but clinically distinct chronic inflammatory arthropathies.

### EXAMPLE 23

### NORI Expression Induced by Pro-inflammatory Mediators

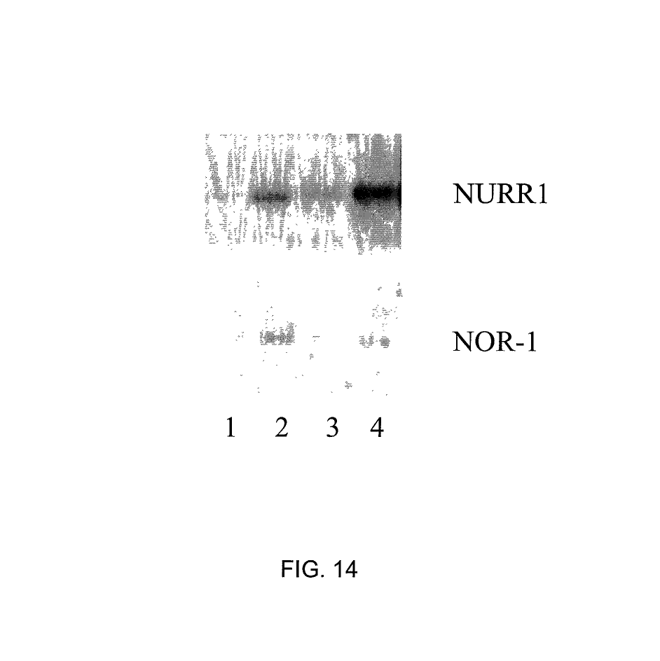

The pathological importance of the NURR transcription factors in inflammatory joint disease was further examined by determining the ability of proinflammatory agonists to regulate NOR1 transcript levels in primary synoviocytes. (FIG. 14). Expression levels of NOR1 and NURR1 mRNA increased compared to the untreated synoviocytes upon treatment with TNFα. However, PGE₂ had the most potent and sustained effect in stimulating NURR1 MRNA in these cells but NORI displayed only a moderate increase in mRNA expression levels.

Northern analysis of primary RA synoviocytes were untreated (lane 1) or treated for 1 hour with TNFα (lane 2), IL-1β (lane 3), and PGE₂ (lane 4). TNFα and IL-1β were used at a concentration of 10 ng/mL and PGE₂ was at a concentration of 10-6 M.

### EXAMPLE 24

### Methods for Tissue Collection

Synovium was obtained from the knee by arthroscopy following informed consent from patients diagnosed with RA, psoriatic arthritis (PsA) or sarcoid arthritis (SA). At the time of biopsy all patients had active disease of recent onset (<12 months). All patients attended the Early Arthritis Clinic at St. Vincent's University Hospital, Dublin, Ireland. Patients were excluded if they were being treated with or had ever taken long term disease modifying drugs. Histologically normal synovium was obtained from patients undergoing lower limb amputation. Human myometrial tissue expressing CRH-R1 mRNA was acquired from a premenopausal patient undergoing hysterectomy, while normal human cerebral cortex cDNA (Gene pool, Invitrogen, Groningen, The Netherlands) served as a control for CRH-R2.

### EXAMPLE 25

### Methods for Synoviocyte Cell Culture and Transient Transfection

Synovial tissue was treated for 4 h with 1 mg/ml collagenase (type I; Worthington Biochemical, Freehold, N.J.) in RPMI at 37° C. in 5% CO₂. Dissociated cells were plated in RPMI supplemented with 10% fetal calf serum (GibcoBRL, Paisley, UK), penicillin (100 U/ml), streptomycin (100 U/ml) and fungizone (0.25 μg/ml). Synoviocyte cells were found to be morphologically homogeneous fibroblast-like cells and were used between the third and seventh passages (Ben-Av et al., 1995). Twenty-four hours before transfection, 1×10⁶ synoviocyte cells were plated in 25 cm² dishes and allowed to attach. Endotoxin free-DNA (3 g CMV-β-galactosidase or 3 μg -666/+111 hCRH CAT (Murphy and Conneely, 1997) was added to 35 μl lipotaxi reagent (Stratagene, Cambridge, UK) in a volume of 900 μl RPMI (serum free) and incubated at room temperature for 30 min. RPMI (1.5 ml) was added to the transfection mixture, transferred onto the cells and incubated for 6 h at 37° C. in 5% CO₂. An equal volume of media was added, and the cells were incubated overnight at 37° C. in 5% CO₂. The DNA-lipotaxi containing media was replaced, and the cells were left untreated or treated with 10 ng/ml IL-1β, IL-6, TNFα, 1 μM PGE₂ or MoCM and incubated for a further 24 h. Chloramphenicol acetyl transferase (CAT) levels were measured using a CAT ELISA (Boehringer Mannheim, Germany). Protein concentrations were determined by the Bradford assay (BioRad, Richmond, Calif.). For β-galactosidase assays cells were washed with cold phosphate buffered saline (PBS), fixed with cold 0.5% glutaraldehyde and washed twice with PBS before incubation with staining solution (1M MgCl₂, 5M NaCL, 0.5M HEPES pH7.3, 30 mM potassium ferricyanide, 30 mM potassium ferrocyanide and 2% 5-bromo-4-chloro-3-indoyl-β-galactopyranoside) for 12 h at 37° C.

### EXAMPLE 26

### Isolation and Culture of Synovial Membrane Endothelial Cells (SMECs)

Primary SMECs are microvascular endothelial cells, which makes them ideal for studies on angiogenesis, a microvascular process. Total knee synovium was obtained at the time of arthroplasty and treated as described above. The digested tissue was passed through a 50-mesh cell dissociation sieve (Sigma Chemical Company, St. Louis, Mo.) and resuspended in 500 μl of cold PBS/0.1% BSA. 1×10⁷ of CD31 (PECAM-1: 9G11) coated Dynabeads, (Dynal A.S. Oslo, Norway) were added to the sample for 45 minutes and placed in a magnetic particle concentrator. The isolated endothelial cells were cultured directly in modified EBM-2MV media (Biowhittaker Inc. Clonetics Products, San Diego, Calif.). Endothelial cells were identified morphologically and by Factor VIII and CD31 expression.

### EXAMPLE 27

### Reverse Transcriptase-polymerase Chain Reaction (RT-PCR)

Total RNA was isolated (RNeasy, Qiagen, UK) from freshly obtained synovial biopsies or cultured synoviocytes. Complimentary DNA (cDNA) was prepared by reverse transcription of 1 μg of each RNA sample using 200U Superscript II (GibcoBRL, Paisley, UK), 100 mM dithiothreitol, 40 U RNasin, 1.25 mM each of DNTP and 120 ng random hexamers at 37° C. for 1 h. PCR were performed in 50 μl volumes containing 2 μl of cDNA reaction mixture, 1.25 mM each of dNTP, 100 ng of each primer, 2 mM MgCL₂ and 2.5 U AmpliTaq-Gold (Perkin Elmer, Brachburg, NJ). For PCR amplification the primer pair, sense 5′-CAATCGAGCTGTCAAGAGAGC3′ (SEQ ID NO:144) and antisense 5′-GGAAGAAATCCAAGGGCTGAG-3′ (SEQ ID NO:145) were used to amplify human CRH. The primer pair, sense 5′CCACCCATGGCAAATTCCATGGCA-3′ (SEQ ID NO:146) and antisense 5′TCTAGACGGCAGGTCAGGTCCACC-3′ (SEQ ID NO:147), were used to amplify GAPDH. Both primer pairs flanked intronic sequences. The conditions for amplification were 97° C. for 1 min, 60° C. for 1 min and 72° C. for 1 min. It was confirmed that 35 cycles for the CRH and 25 cycles for the GAPDH primer pairs ensured the PCR reactions had not reached the plateau phase of amplification. The PCR products were confirmed by Southern analysis. Autoradiographic intensity was quantitated using an imaging densitometer.

Primers for human CRH-R1 were designed to distinguish α and β isoforms. CRH-R1β contains an 87 base pair insertion at the 5′ region resulting in primers yielding a 333 bp product for CRH-R1α or 420 bp product corresponding to CRH-R1β. Primer pairs flanked intronic sequences and were designed as follows: sense primer 5′-GCC CTG CCC TGC CTT TTT CTA -3′ (SEQ. ID NO:148) and antisense primer 5′-GCT CAT GGT TAG CTG GAC CA-3′ (SEQ. ID NO:149) corresponding to positions 235-255 and 549-568 respectively for CRH-R1α, or corresponding to positions 198-219 and 599-618 respectively for CRH-R1β. Primers for human CRHR2 were used to amplify a 781 bp product identifying receptor isoforms α and β sense primer 5′-GCT GGC CCC GCA GCG CTG CC-3′ (SEQ. ID NO:150) and antisense primer 5′-CCT CAC TGC CTT CCT GTA CT-3′ (SEQ. ID NO:151), corresponding to positions 149-169 and 911-930 respectively. GAPDH primers were designed to produce a 635 bp product: sense primer 5′-CCACCCATGGCAAATTCCATGGCA-3′ (SEQ. ID NO:146) and antisense primer 5′-TCTAGACGGCAGGTCAGGTCCACC3′ (SEQ. ID NO:147). PCR were performed in 50 l volumes containing 2 μl of cDNA, 1.25 mM each of dNTP, 2.5 U AmpliTaq Gold with 1× PCR buffer II (Perkin Elmer, Brachburg, N.J.), 1.0 mM MgCl₂ (CRH-R1, GAPDH), 1.5 mM MgCl₂ (CRHR2), 200 ng of sense and antisense primers (CRH-R1), and 100 ng of sense and antisense primers (CRH-R2, GAPDH). The conditions for amplification were denaturation at 94° C. for 1 minute, primer annealing from 62-58° C. for 1 minute (CRH-R1 and GAPDH) and from 64-60° C. for 1 minute (CRH-R2) and extension at 72° C. for 1 minute. Each PCR sample underwent a 35-cycle amplification which ensured that the reactions had not reached the plateau phase of amplification. PCR products generated were electrophoresed on a 1.5% agarose gel and visualized. The identity of PCR products was confirmed by sequencing.

### EXAMPLE 28

### Methods for Northern Blot Analysis

Total RNA was isolated from cultured primary synoviocytes or synovial explants. RNA was quantitated by UV absorption and 10 μg of total RNA were electrophoresed on a standard northern gel and transferred to nylon membrane (BioRad, Richmond, Calif.). NURR1 and NUR77 cDNA probes (Murphy and Conneely, 1997), spanning the amino terminal region to avoid cross-hybridization, were radiolabeled to a high specific activity using [α-³²P] dCTP and a random primer labeling system (Promega, Madison, Wis.). Blots were exposed to film at −80° C. using intensifying screens and autoradiographic intensity was quantitated using an imaging densitometer.

### EXAMPLE 29

### Methods for Immunohistochemistry

Synovial tissue sections (6 μm) were placed on glass slides coated with 2% aminopropyl-triethoxy-silane or 1% paraformaldehyde. Synoviocytes grown on apyrogenic glass coverslips were treated with methanol for 15 min before staining. The primary antibody (1:100 dilution) for NURR1 (Santa Cruz Biotechnology, Santa Cruz, Calif.) was a rabbit polyclonal antibody mapping to the amino terminus of human and rat NURR1. The primary antibody (1:75 dilution) for CRH receptor I (C-20) was a goat polyclonal antibody (Santa Cruz Biotechnology, Santa Cruz, Calif.) raised against a peptide mapping at the carboxy terminus of the human CRH-receptor type 1 (CRH-R1). C-20 reacts with both CRH-R1 and a second CRH receptor subtype designated CRH-R2. Specific CRH-R1 staining was achieved by eliminating the CRH-R2 activity of C-20 by pre-absorbing the antibody with an excess of CRH-R2 specific synthetic peptide (N-20-P, 100 μg/0.5 ml; Santa Cruz Biotechnology, Santa Cruz, Calif.). The primary antibody for CRH-R2 (N-20) was a polyclonal antibody (200 μg/ml, Santa Cruz Biotechnology, Santa Cruz, Calif.) raised against a peptide corresponding to the amino terminus of CRH-R2. Primary antibodies were diluted 1:100 in 0.6M NaCl and incubated on sections at 37° C. Following 2 h incubation with the primary antibody, a biotinylated secondary antibody (1:500; Vector laboratories, Burlingame, Calif.) was spotted on sections, followed by the avidin-biotin-peroxidase complex (ABC kit, Vectastain, Burlingame, Calif.). For negative controls, each primary antibody was preabsorbed with its specific synthetic peptide (20011 g/ml; Santa Cruz Biotechnology, Santa Cruz, Calif.).

### EXAMPLE 30

### Preparation of Nuclear Extracts and Electrophoretic Mobility Shift Assays (EMSAs)

Nuclear protein extracts were prepared as previously described (Murphy and Conneely, 1997). For the EMSAs, 1 μg of nuclear extract was incubated for 20 mins in the presence of 20 mM Hepes (pH 7.9), 5 mM MgCl_{2, 20}% glycerol, 100 mM KCL, 0.2 mM EDTA, 8% Ficoll, 600 mM KCL, 500 ng/μl poly (deoxyinosinicdeoxycytidylic) acid, 50 mM dithiothreitol (DTT) and [α-³²P]-dCTP labeled doublestranded oligonucleotide. For supershifts experiments, antibodies to NURR1 (Santa Cruz Biotechnology, Santa Cruz, Calif.) were added following the initial 20-minute incubation, then incubated for an additional 20 minutes. The samples were electrophoresed through a 5.5% non-denaturing polyacrylamide gel in 0.5× TrisBorate-EDTA (TBE) buffer. For competition studies, the reaction was performed as described with the indicated concentrations of unlabeled probe. The oligonucleotide CRH NBRE 5′-GATGGTAAGAAGGTCAACGG-3′ (SEQ ID NO:152) was used in the mobility shift assay.

### EXAMPLE 31

### Methods for Statistical analysis

Data are expressed as mean values +/− SE. Comparisons between normal synovium and synovium obtained from patients diagnosed with RA, PsA or SA were made using the Student's t test for unpaired values. Comparisons between treatment were made using Student's t test for paired values.

### EXAMPLE 32

### Method for Dual-labeled Immunofluorescence

Tissue sections or isolated cell cultures were incubated in diluted normal rabbit serum (Vector Laboratories, Burlingame, Calif., USA). CRH-R1 (C-20) polyclonal antibody was diluted 1:10 in 10% normal human serum followed by the addition of biotinylated anti-goat secondary antibody (1:500, Vector Laboratories, Burlingame, Calif.). Sections were incubated in diluted normal goat serum and incubated in a 1:10 dilution of the second primary antibody, a monoclonal mast cell marker: mouse anti-human tryptase (AA1, Accurate Chemical Scientific Corp. Westbury, N.Y., USA). Goat anti-mouse IgGl FITC conjugated antibody (1:50, 1 mg/ml, Southern Biotechnology Associates, Inc. Birmingham Ala., USA) was added followed by Cy3 fluorochrome conjugated monoclonal mouse anti-biotin antibody (BN-34, 1:100, Sigma Chemical Company, St. Louis, Mo., USA). Slides were mounted in DAKO® fluorescent mounting medium (Dako Corporation, Carpinteria, Calif., USA). Isotype matched non-immune IgG were included as controls for each of the primary antibodies and the primary antibodies were sequentially omitted to ensure specific staining for each. A similar protocol was employed for Factor VIII (1:50, F8/86, Dako Corporation, CA, USA) staining of isolated SMECs.

### EXAMPLE 33

### Method to Screen for Antagonist of NURR Subfamily Members

A transcription assay is carried out in the presence and absence of the test agent. A cell harboring a NURR subfamily member and a marker sequence operatively linked to a promoter, wherein the promoter is regulated either directly or indirectly by a NURR subfamily member, is administered a test agent. A decrease in expression of the marker sequence in the presence of the agent compared to that in its absence indicates that the agent interferes with a NURR subfamily member/ligand interaction. The interference is direct or indirect. A skilled artisan recognizes that the marker sequence is any marker sequence which reflects indirectly or directly its expression level. Furthermore, the transcript produced by the expression of the marker sequence or the gene product of the expression is detected. Examples of marker sequences are well known in the art and include, chloramphenicol acetyl transferase, β-galactosidase, β-glucuronidase, green fluorescent protein, blue fluorescent protein, luciferase, and so forth. As provided in the Examples herein, dexamethasone reduces pro-inflammatory mediator expression of NURR1 and thus acts as a NURR subfamily member antagonist in the presence of pro-inflammatory mediators.

Thus, there is a method of screening for a compound that interferes with interaction of a NURR subfamily polypeptide with a ligand, comprising the steps of introducing to a cell a test agent, wherein the cell comprises a marker sequence, wherein the expression of the marker sequence is regulated by said NURR subfamily member; and measuring the expression level of the marker sequence, wherein when said expression of said marker sequence is reduced following said introduction, said test agent is the compound that interferes with the interaction of the NURR subfamily polypeptide with the ligand.

In another specific embodiment, the method identifies a compound for the treatment of an inflammatory immune disease, comprising the steps of introducing to a cell a test agent, wherein the cell comprises a marker sequence, wherein the expression of the marker sequence is regulated by said NURR subfamily member; and measuring the expression level of the marker sequence, wherein when said expression of said marker sequence is reduced following said introduction, said test agent is the compound for the treatment of said inflammatory immune disease.

### Patents

U.S. Pat. No. 3,817,837

U.S. Pat. No. 3,850,752

U.S. Pat. No. 3,939,350

U.S. Pat. No. 3,996,345

U.S. Pat. No. 4,277,437

U.S. Pat. No. 4,275,149

U.S. Pat. No. 4,366,241

U.S. Pat. No. 3,376,110

U.S. Pat. No. 4,676,980

U.S. Pat. No. 4,797,368

U.S. Pat. No. 4,816,567

U.S. Pat. No. 5,139,941

U.S. Pat. No. 5,662,298

U.S. Pat. No. 5,662,299

U.S. Pat. No. 5,670,488

U.S. Pat. No. 6,147,275

U.S. Pat. No. 6,127,399

U.S. Pat. No. 5,821,337

WO 91/00360

WO 92/200373

EP 03089

### PUBLICATIONS

All patents and publications mentioned in the specification are indicative of the level of those skilled in the art to which the invention pertains. All patents and publications are herein incorporated by reference to the same extent as if each individual publication was specifically and individually indicated to be incorporated by reference.

Aguilera G, Millan M A, Hauger R L, Catt K J. 1987. Corticotrophin-releasing factor receptors: distribution and regulation in brain, pituitary, and peripheral tissues. Ann. New York Acad. Sci. 512:48-66.

Angel P, Baumann I, Stein B, Delius H, Rahmsdorf H J, Herrlich P. 1987. 12-O-tetradecanoyl-phorbol-13-acetate induction of the human collagenase gene is mediated by an inducible enhancer element located in the 5′-flanking region. Mol. Cell. Biol. 7:2256-2266

Arbiser J L, Karalis K, Viswanathan A, Koike C, Anand-Apte B, Flynn E, Zetter B, Majzoub J A. 1999. Corticotropin-releasing hormone stimulates angiogenesis and epithelial tumor growth in the skin. J Invest Dermatol. 113:838-842.

Ben-Av P, Crofford LJ, Wilder RL Hla T. 1995. Induction of vascular endothelial growth factor expression in synovial fibroblasts by prostaglandin E and interleukin-1: a potential mechanism for inflammatory angiogenesis. FEBS Lett. 372:83-87.

Bucala R, Ritchlin C, Winchester R, Cerami A. 1991. Constitutive production of inflammatory and mitogenic cytokines by rheumatoid synovial fibroblasts. J. Exp. Med. 173:569-574.

Cato ACB, Wade E. 1996. Molecular mechanisms of anti-inflammatory action of glucocorticoids. Bioessays. 18:371-380.

Crofford L J, Sano H, Karalis K, Friedman T C, Epps H R, Remmers E F, Mathem P, Chrousos G P, Wilder R L. 1993. Corticotropin releasing hormone in synovial fluids and tissues of patients with rheumatoid arthritis and osteoarthritis. J. Immunol. 151:1587-1592.

Crofford L J, Wilder R L, Ristimaki A P, Sano H. 1994. Cyclooxygenase-1 and -2 expression in rheumatoid synovial tissue. J.Clin.Invest. 93:1095-1101

Crofford L J, Sano H, Karalis K, Webster E L, Goldmuntz E A, Chrousos GP, Wilder RL. 1992. Local secretion of corticotrophin releasing hormone in the joints of Lewis rats with inflammatory arthritis. J. Clin. Invest. 90:2555-2564.

Davis I J, Lau L F. 1994. Endocrine and neurogenic regulation of the orphan nuclear receptors NUR77 and NURR1 in the adrenal glands. Mol. Cell Biol. 14:3469-3483.

Evans, R. 1988. The steroid and thyroid hormone receptor superfamily. Science. 240:889-895.

Fearon U, Reece R, Jack A, Blythe D, Emery P, Veale D. 1998. Co-localisation of leukaemia inhibitory factor with stat1α protein, and increased ACTH expression, in very early rheumatoid arthritis synovium. Br. J. Rheumatol. 37: S33

Firestein G S, Manning A M. 1999. Signal transduction and transcription factors in rheumatic disease. Arthritis Rheum. 42:609-621

Guardiola-Diaz H M, Boswell C, Seasholtz A F. 1994. The cAMP-responsive element in the corticotropin-releasing hormone gene mediates transcriptional regulation by depolarization. J. Biol. Chem. 269:14784-14789.

Hazel T G, Nathans D, Lau L F. 1988. A gene inducible by serum growth factors encodes a member of the steroid and thyroid hormone receptor superfamily. Proc. Natl. Acad. Sci. USA. 85:8444-8448.

Hedvat C V, Irving S G. 1995. The isolation and characterization of MINOR, a novel mitogen-inducible nuclear orphan receptor. Mol. Endocrinol. 9:1692-1700.

Karalis K, Sano H, Redwine J, Listwak M, Wilder R L, Chrousos G P. 1991. Autocrine or paracrine inflammatory actions of corticotropin releasing hormone in vivo. Science. 254:421-423.

Karalis K, Kantopoulos E, Muglia L J, Majzoub J A. 1999. Corticotropin-releasing hormone deficiency unmasks the proinflammatory effect of epinephrine. Proc. Natl. Acad. Sci. USA 96:7093-7097.

Kehayova, P. D., Bokinsky, G. E., Huber, J. D., Jain, A. (1999) A caged hydrophobic inhibitor of carbonic anhydrase II. J. Amer. Chem. Soc., 1: 187-188.

Law S W, Conneely O M, DeMayo F J, O'Malley B W. 1992. Identification of a new brain specific transcription factor, Nurr1. Mol. Endocrinol. 6:2129-2135.

Labrie F, Veilleux R, Lefevre G, Coy D H, Sueiras-Diaz J, Schally A. 1982. Corticotropin-releasing factor stimulates accumulation of adenosine 3′,5′monophosphate in rat pituitary corticotrophs. Science. 216:1007-1008

MacNaul K L, Chartrain N, Lark M, Tocci M J, Hutchinson N I. 1990. Discoordinate expression of stromelysin, collagenase, and tissue inhibitor of metalloproteinases-1 in rheumatoid synovial fibroblasts. Synergistic effects of interleukin-1 and tumor necrosis

Mages H W, Rilke O, Bravo R, Senger G, Kroczek R A. 1994. NOT, a human immediate-early response gene closely related to the steroid/thyroid hormone receptor NAK1/TR3. Mol. Endocrinol. 8:1583-1591.

Maruyama K, Tsukada T, Bandoh S, Sasaki K, Ohkura N, Yamaguchi K. 1995. Expression of NOR-1 and its closely related members of the steroid/thyroid hormone receptor superfamily in human neuroblastoma cell lines. Cancer Lett. 96:117-122.

Milbrandt J. 1988. Nerve growth factor induces a gene homologous to the glucocorticoid receptor gene. Neuron. 1:183-188

Murphy E P, Conneely O M. 1997. Neuroendocrine regulation of the Hypothalamic Pituitary Adrenal Axis by the NURR1/NUR77 subfamily of nuclear receptors. Mol. Endocrinol. 1:39-47.

Murphy E P, Dobson A D W, Keller C H, Conneely O M. 1995. Differential regulation of transcription by the NURR1/NUR77 subfamily of nuclear transcription factors. Gene Express. 5:169-179.

Nakai A, Kartha S, Sakurai A, Toback F G, DeGroot L J. 1990. A human early response gene homologous to murine nur77 and rat NGFI-B, and related to the nuclear receptor superfamily. Mol. Endocrinol. 4:1438-1443.

Nishioka T, Kurokawa H, Takao T, Kumon Y, Nishiya K, Hashimoto K. 1996. Differential changes of corticotropin releasing hormone concentrations in plasma and synovial fluids of patients with rheumatoid arthritis. Endocrine J. 43:241-247.

Ohkura N, Hijikuro M, Yamamoto A, Miki K. 1994. Molecular cloning of a novel thyroid/steroid receptor superfamily gene from cultured rat neuronal cells. Biochem. Biophys. Res. Comm. 205:1959-1965

O'Malley B W, Conneely O M. 1992. Minireview: Orphan receptors: in search of a unifying hypothesis for activation. Mol. Endocrinol. 6:1359-1361.

Parkes D, Rives S, Lee S, Rivier C, Vale W. 1993. Corticotropin-releasing factor activates c-fos, NGFI-B, and corticotropin-releasing factor gene expression within the paraventricular nucleus of the rat hypothalamus. Mol. Endocrinol. 7:1357-1367.

Perrin M, Donaldson C, Chen R, Blount A, Berggren T, Bilezikjian L, Sawchenko P, Vale W. 1995. Identification of a second corticotrophin-releasing factor receptor gene and characterization of a cDNA expressed in heart. Proc. Natl. Acad. Sci. 92:2969-2973.

Philips A, Lesage S, Gingras R, Maira M H, Gauthier Y, Hugo P, Drouin J. 1997. Novel dimeric Nur77 signaling mechanism in endocrine and lymphoid cells. Mol. Cell Biol. 17:5946-5951.

Philips A, Maira M, Mullick A, Chamberland M, Lesage S, Hugo P, Drouin J. 1997. Antagonism between Nur77 and glucocorticoid receptor for control of transcription. Mol. Cell. Biol. 17:5952-5959.

Rivera, V. M., Wang, X., Wardwell, S., Courage, N. L., Volchuk, A., Keenan, T., Holt, D. A., Gilman, M., Orci, L., Cerasoli, F., Rothman, J. E., Clackson, T. (2000) Regulation of protein secretion through controlled aggregation in the endoplasmic reticulum. Science, 287: 826-836.

Ryseck R P, MacDonald-Bravo H, Mattei M G, Ruppert S, Bravo R. 1989. Structure, mapping and expression of a growth factor inducible gene encoding a putative nuclear hormonal binding receptor. EMBO J. 8:3327-3335.

Scearce L M, Laz T M, Hazel T G, Lau L F, Taub R. 1993. RNR-1, a nuclear receptor in the NGFI-B/Nur77 family that is rapidly induced in regenerating liver. J. Biol. Chem. 268:8855-8861.

Subramanian, A., Ranganathan, P., Diamond, S. L. (1999) Nuclear targeting peptide scaffolds for lipofection of nondividing mammalian cells, Nat. Biotech. 17: 873-877.

Subramanian, G., Hjelm, R. P., Deming, T. J., Smith, G. S., Li, Y., Safinya, C. R. (2000) Structure of complexes of cationic lipids and poly (glutamic acid) polypeptides: a pinched lamellar phase. J. Amer. Chem. Soc. 122: 26-34.

Theoharides T C, Singh L K, Boucher W, Pang X, Letroumeau R, Webster E, Chrousos G. 1998. Corticotropin releasing hormone induces skin mast cell degranulation and increased vascular permeability, a possible explanation for its pro-inflammatory effects. Endocrinology. 139:403-413.

Turnbull A V, Rivier C L. 1999. Regulation of the hypothalamic-pituitary-adrenal axis by cytokines: actions and mechanisms of action. Physiol. Rev. 79:1-71

Uhlar C M, Grehan S, Steel D M, Steinkasserer A, Whitehead AS. 1997. Use of the acute phase serum amyloid A2 (SAA2) gene promoter in the analysis of pro- and antiinflammatory mediators. Differential kinetics of SAA2 promoter induction by IL-1b and TNF-α compared to IL-6. J. Immunol. Meth. 203:123-130.

Veale D, Yanni G, Rogers S, Barnes L, Bresnihan B, FitzGerald O. 1993. Reduced synovial membrane macrophage numbers, ELAM-1 expression, and lining hyperplasia in psoriatic arthritis as compared with rheumatoid arthritis. Arthritis Rheum 36: 893-900.

Vale W, Speiss J, Rivier C, Rivier R J.1981. Characterisation of a 41-residue ovine hypothalamic peptide that stimulates secretion of corticotropin and beta-endorphin. Science. 213:1394-1396.

Wilson, T E, Mouw A R, Weaver C A, Milbrandt J, Parker K L. 1993. The orphan nuclear receptor, NGFI-B regulates expression of the gene encoding steroid-21-hydroxylase. Mol. Cell. Biol. 13:861-868.

Wilson T E, Fahrner T J, Johnston M, Milbrandt J. 1991. Identification of the DNA binding site for NGFI-beta by genetic selection in yeast. Science. 252:1296-1300.

Watson M A, Milbrandt J. 1989. The NGFI-B gene, a transcriptionally inducible member of the steroid receptor gene superfamily: genomic structure and expression in rat brain and seizure induction. Mol. Cell. Biol. 9:4213-4219.

Webster E L, Torpy D J, Elenkov I L, Chrousos G P. 1998. Corticotropin releasing hormone and inflammation. Ann.New York Acad. Sci. 840:21-32.

One skilled in the art readily appreciates that the present invention is well adapted to carry out the objectives and obtain the ends and advantages mentioned as well as those inherent therein. Sequences, methods, treatments, pharmaceutical compositions, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. An antagonist to inhibit transcriptional activity of a nuclear receptor polypeptide, wherein the polypeptide comprises a NURR subfamily amino acid sequence.

2. The antagonist of claim 1, wherein the NURR subfamily amino acid sequence is selected from the group consisting of SEQ ID NO:33, SEQ ID NO:64 and SEQ ID NO:91.

3. The antagonist of claim 1, wherein the antagonist inhibits arthritis.

4. The antagonist of claim 1, wherein the antagonist inhibits joint inflammation.

5. An antagonist to inhibit transcriptional activity of a nuclear receptor polypeptide, wherein the polypeptide comprises a NURR1 amino acid sequence.

6. The antagonist of claim 5, wherein the NURR1 amino acid sequence is SEQ ID NO:33.

7. The antagonist of claim 5, wherein the antagonist inhibits arthritis.

8. The antagonist of claim 5, wherein the antagonist inhibits jointinflammation.

9. A method of treating an organism for an inflammatory immune disease comprising the step of reducing expression of a NURR subfamily nucleic acid sequence.

10. A method of claim 9, wherein said NURR subfamily nucleic acid sequence is selected from the group consisting of SEQ ID NO: 1, SEQ ID NO:47 and SEQ ID NO:76.

11. A method of of treating an organism for an inflammatory immune disease comprising the step of reducing expression of a NURR subfamily nucleic acid sequence of SEQ ID NO: 1.

12. The method of claim 9, wherein said inflammatory immune disease is selected from the group consisting of a chronic inflammatory joint disease, ulcerative colitis and thyroiditis.

13. The method of claim 10, wherein said chronic inflammatory joint disease is arthritis.

14. The method of claim 13, wherein said arthritis is selected from the group consisting of rheumatoid arthritis, psoriatic arthritis and sarcoid arthritis.

15. A method of treating an organism for an inflammatory immune disease comprising the step of reducing the level of a polypeptide comprising a NURR subfamily amino acid sequence.

16. The method of claim 15, wherein the NURR subfamily amino acid sequence is selected from the group consisting of SEQ ID NO:33, SEQ ID NO: 64 and SEQ ID NO:91.

17. The method of claim 15, wherein the NURR subfamily amino acid sequence is SEQ ID NO:33.

18. The method of claim 15, wherein said reduction of said polypeptide comprises inhibiting amino acid synthesis of a sequence comprising SEQ ID NO:33.

19. The method of claim 15, wherein said inflammatory immune disease is selected from the group consisting of a chronic inflammatory joint disease, ulcerative colitis and thyroiditis.

20. The method of claim 19, wherein said chronic inflammatory joint disease is arthritis.

21. The method of claim 20, wherein said arthritis is selected from the group consisting of rheumatoid arthritis, psoriatic arthritis and sarcoid arthritis.

22. A method of treating an organism for an inflammatory immune disease comprising the step of inhibiting transcriptional activity of a NURR subfamily member.

23. The method of claim 22, wherein the amino acid sequence of the NURR subfamily member is selected from the group consisting of SEQ ID NO:33, SEQ ID NO:64 and SEQ ID NO:91.

24. The method of claim 22, wherein the amino acid sequence of the NURR subfamily member is SEQ ID NO:33.

25. The method of claim 22, wherein said inflammatory immune disease is selected from the group consisting of a chronic inflammatory joint disease, ulcerative colitis and thyroiditis.

26. The method of claim 25, wherein said chronic inflammatory joint disease is arthritis.

27. The method of claim 26, wherein said arthritis is selected from the group consisting of rheumatoid arthritis, psoriatic arthritis and sarcoid arthritis.

28. A method of screening for a compound that interferes with interaction of a NURR subfamily polypeptide with a ligand, comprising the steps of:
introducing to a cell a test agent, wherein the cell comprises a marker sequence, wherein the expression of the marker sequence is regulated by said NURR subfamily member; and
measuring the expression level of the marker sequence, wherein when said expression of said marker sequence is reduced following said introduction, said test agent is the compound that interferes with the interaction of the NURR subfamily polypeptide with the ligand.

29. As a composition of matter, the compound obtained by the method of claim 28.

30. The method of claim 28, wherein said NURR subfamily polypeptide is a sequence selected from the group consisting of SEQ ID NO:33, SEQ ID NO:64 and SEQ ID NO:91.

31. The method of claim 28, wherein said NURR subfamily polypeptide is a sequence of SEQ ID NO:33.

32. A method of identifying a compound for the treatment of an inflammatory immune disease, comprising the steps of:
introducing to a cell a test agent, wherein the cell comprises a marker sequence, wherein the expression of the marker sequence is regulated by said NURR subfamily member; and
measuring the expression level of the marker sequence, wherein when said expression of said marker sequence is reduced following said introduction, said test agent is the compound for the treatment of said inflammatory immune disease.

33. A pharmacologically acceptable composition, comprising:
the compound obtained by the method of claim 32; and
a pharmaceutical carrier.

34. The method of claim 32, further comprising:
dispersing the compound in a pharmaceutical carrier; and
administering a therapeutically effective amount of said compound in said
carrier to an individual having said inflammatory immune disease.

35. The method of claim 32, wherein said inflammatory immune disease is in ajoint.

36. A method of treating an organism for an inflammatory immune disease comprising the step of reducing expression of a corticotropin releasing hormone receptor nucleic acid sequence.

37. A method of claim 36, wherein said reducing of corticotropin releasing hormone receptor expression comprises inhibiting synthesis of a nucleic acid sequence of SEQ ID NO:104.

38. A method of treating an organism for an inflammatory immune disease comprising the step of reducing the level of a corticotropin releasing hormone receptor amino acid sequence.

39. The method of claim 38, wherein said reduction of the corticotropin releasing hormone receptor amino acid level comprises inhibiting amino acid synthesis of a sequence comprising SEQ ID NO: 124.

40. A method of treating an organism for an inflammatory immune disease comprising the step of inhibiting the transcriptional activity of a sequence of SEQ ID NO: 124.

41. An antagonist to decrease expression of a NURR subfamily member.

42. The antagonist of claim 41, wherein said antagonist is dexamethasone.
